(19) United States
(12) Patent Application Publication
(10) Pub. No.: US 2018/0261305 A1
(43) Pub. Date: Sep. 13, 2018

(54) Clinical Trial Data Analyzer

(71) Applicant: Emmes Software Services, LLC, Rockville, MD (US)

(72) Inventors: Anne LINDBLAD, Middleburg, VA (US); Zorayr Mikael Manukyan Zakaryan, Lexington, MA (US)

(73) Assignee: Emmes Software Services, LLC, Rockville, MD (US)

(21) Appl. No.: 15/868,732

(22) Filed: Jan. 11, 2018

Related U.S. Application Data
(60) Provisional application No. 62/561,265, filed on Sep. 21, 2017.
(60) Provisional application No. 62/469,467, filed on Mar. 9, 2017.

(51) Int. Cl. G16H 10/20; G06N 7/00

(52) U.S. Cl. CPC G16H 10/20; G06N 7/005

(57) ABSTRACT

Systems, devices, and methods for analyzing clinical trial data by storing the clinical trial data in a trial monitoring data object; using multiple metric functions to generate multiple metric values that each indicate a statistical attribute of a variable of a patient; creating an analytical data object that stores the metric values and contents of the trial monitoring object; determining, for each metric function “f” and each variable “v”, a risk score S_{f,v}(u) associated with each clinical unit “u”; creating a risk data object that stores the risk scores “S_{f,v}(u)” and contents of the analytical data object; and displaying a graphical user interface that permits a user to visualize and recognize patterns in the data by interacting with a three-dimensional matrix that selectively displays markers for risk scores based on a risk threshold, where selecting a marker corresponding to a risk score allows for further investigation of the corresponding unit.

### FIELD OF INVENTION

The present invention relates generally to clinical trial monitoring systems, and more specifically to systems and methods for analyzing clinical trial data to identify potentially erroneous or untrustworthy data resulting from flawed, defective or incomplete data collection and recording practices resulting from intentional or unintentional noncompliance, negligence, fraud or misconduct.

### BACKGROUND

A clinical trial (sometimes referred to as a clinical study) is a research or investigation program conducted to investigate and record data associated with a disease or condition or testing the efficacy and/or impact on human patients of a pharmaceutical product, a medical device or treatment regimen for a medical disease or condition. A clinical trial “participant” is a person, such as a clinical trial monitor, a doctor, a nurse, a patient or volunteer, that participates in a clinical trial, clinical research or investigation program. A clinical trial “subject” typically refers to a patient or volunteer participating in a clinical trial. For the sake of clarity and avoidance of confusion that might be caused by using the words “subject,” “volunteer” and “patient” interchangeably, clinical trial “subjects” and clinical trial “volunteers” will hereinafter be referred to in this disclosure as “patients.” Therefore, for purposes of this disclosure, the word “patient” should be understood to refer to a clinical trial subject, a clinical trial volunteer, or both. A clinical trial “site” is typically a physical location (i.e., a geographical location such as a room, a building, a country, a region of the world, etc.) where a clinical trial occurs. A clinical trial site may also include a virtual location (such as a computer system, computer network or online website) where a trial participant is enrolled or where data are collected and stored for a clinical trial. A clinical trial “unit,” as used herein, may refer to a clinical trial site, a geographic region, a collection of trial sites, a clinical trial participant (as defined above) at a clinical trial site, a device used at a clinical trial site (e.g., a stethoscope, a blood pressure or heart rate monitor, a laptop or desktop computer system), or any other person, entity or device associated with measuring, recording and/or storing data generated for a clinical trial.

Clinical trial data may include enrollment data, clinical data, adverse event data, other data, or some combination of enrollment, clinical, adverse event and other data. Enrollment data refers to a collection of data from a clinical trial that includes, but is not limited to, participant demographic data (e.g., name, address, age, sex, physical condition, etc.), data on the time and place of enrollment and/or subsequent visits of a trial participant, as well as treatments and interventions planned for or administered to a patient (or subject) during a clinical trial.

Clinical data from a clinical trial refers to a collection of measurements taken and recorded for participating patients in the clinical trial, including but not limited to measurements for certain patient-related health variables, such as blood pressure, heart rate and body temperature and intervention response variables, such as disease or condition changes and patient reported outcomes. These measurements are typically taken and recorded by clinical trial participants in the clinical trial. Clinical data may be organized into separate data sets, based for example, on the type of data collected for example lab data, or on the date, time or geographic location that the measurements are taken and recorded, for example when a visit occurred, and/or the date, time or geographic location that adverse events appeared and/or were recorded. The time-organized data sets may include time values that are expressed in absolute times, or alternatively expressed in times that are relative to the dates and times of enrollment.

Adverse event data includes data related to any untoward medical occurrence or condition in a patient who has received the pharmaceutical product or treatment or procedure associated with the clinical trial. An adverse event does not necessarily have a causal relationship with the pharmaceutical product, treatment or procedure. An adverse event can therefore be any unfavorable and unintended sign (including an abnormal laboratory finding), a symptom, or a disease that occurs contemporaneously with the pharmaceutical product, treatment or procedure being investigated, regardless of whether the abnormal finding, symptom or disease is found to be related to the pharmaceutical product, treatment or procedure. Adverse events may be classified as not serious (e.g., a skin irritation or drowsiness) or serious (e.g., life-threatening, requires in-patient or prolonged hospitalization, or death).

Clinical trial units may require monitoring to ensure that data collected “at” these units (if the units are sites) or “by” these units (if the units are people, such as clinical trial monitors) is reliable and accurate. Such monitoring may be aimed at identifying enrollment errors, data collection errors, missing, fraudulent or incomplete data, attempts to hide or conceal missing, fraudulent or incomplete data, etc. Once an issue has been identified and associated with a particular clinical trial unit in a clinical trial, an appropriate action may be recommended and implemented, such as auditing the unit and/or excluding from the clinical trial the problematic data generated by the clinical trial unit, for example. However, the size and dimensionality of the data involved in a clinical trial makes such issue identification a complex and challenging task.

### SUMMARY

Aspects and embodiments of the present invention provide improved devices, methods, and systems for analyzing and displaying clinical data, enrollment data, and adverse event data from multiple clinical trial units of a clinical trial to assist users in discovering, identifying and addressing data that may be unreliable and/or erroneous, and could therefore undermine the integrity of the clinical trial results. Embodiments generate and assign risk scores to multiple variables collected over multiple visits by multiple patients to multiple clinical units, and display an interactive visualization tool representing the variables and assigned risk scores on an interactive display. The interactive visualization tool allows users of the system to conduct root cause analysis on the data to identify and further understand the causes of potential data integrity issues. In some embodiments, a variable with a higher assigned risk score may indicate a higher risk associated with the variable. In other embodiments, a variable with a higher assigned risk score may indicate a lower risk associated with the variable. Among other things, the interactive visualization tool permits a user of an embodiment of the invention to reduce patient dimensionality and/or variable dimensionality in the data, so that the user may observe and compare the data associated with a particular clinical unit against the data associated with all of the other clinical units combined, and thereby more easily detect, identify and analyze statistical anomalies in the data associated with a particular unit. Identifying and analyzing the statistical anomalies in the data helps users detect and address noncompliance, negligence, misconduct or fraudulent data collection practices at that particular unit. In preferred embodiments, a dashboard, summarizing identified issues, is provided to help the user track the statistical anomalies and/or data integrity issues revealed by using the interactive visualization tool to carry out the root cause analysis.

In general, embodiments of the present invention provide a computer system, a method and a user interface for analyzing clinical data, enrollment data and adverse event data collected during a clinical trial. The clinical trial data includes multiple measurements of multiple variables for multiple patients, wherein the data are obtained during multiple visits of the multiple patients to multiple clinical units. The computer system embodiment includes a microprocessor, a display device, a primary memory device for storing an application program comprising instructions executable by the microprocessor, and a secondary memory device for storing a trial monitoring data object, an analytical data object, and a risk data object. The trial monitoring data object stores the original clinical trial data in the form collected at the clinical unit. The analytical data object stores a copy of the trial monitoring data object, as well as a collection of metrics associated with each variable for each patient. Each one of the metrics for each one of the variables is generated by applying one or more metric functions to the measurements of a variable for a patient. The measurements for a patient are typically collected over multiple visits to a clinical unit by the patient. The metric functions are applied to a collection of measurements for a variable for a patient to generate a statistical attribute for the collection of measurements of the variable for that patient. The risk data object stores a copy of the analytical data object and risk scores “S_{f,v}(u)” obtained for each metric function “f”, variable “v”, and unit “u” based on a comparison of a collection of metric values associated with unit “u” with a collection of metric values associated with all other units combined. The microprocessor, when executing the instructions, is configured to display a graphic representation of the risk scores in the user interface on the display device.

In one embodiment, the program instructions in the application program are configured to cause the microprocessor to display on the display device a graphical user interface arranged to assist in analyzing clinical data associated with patients in the situation where each patient visits a single clinical unit within multiple clinical units two or more times without visiting any other clinical unit in the multiple clinical units. In another embodiment, the system may be configured to display a graphical user interface arranged to assist in analyzing clinical data associated with patients in the situation where each patient visits multiple clinical units two or more times during a clinical trial.

In preferred embodiments, the multiple metric functions include a statistical function comprising, for example, a standard deviation, an entropy, a mean value, an average value, a rate of identical measurements, a sum of distances between measured values taken during neighboring visits, an occurrence of similar values among multiple measurements for a variable (within tolerance measurements, or some combination of two or more such statistical functions).

In one embodiment, for a metric function “f1”, a variable “v1”, and a clinical unit “u1”, a risk score “S_{f1,v1}(U1)” indicates a relative strength of differences between the clinical unit “u1” as compared to all other units based on metric values obtained by applying the metric function “f1” to measurements of the variable “v1”. The risk score “S_{f1,v1}(U1)” indicates the likelihood of an error in the measurements of the variable “v1” at the clinical unit “u1” given measurements of the same or related variables at other clinical units.

In one embodiment, the contents of the risk data object are used by the application program and the microprocessor to generate and display on the display device a three-dimensional risk matrix. The three-dimensional risk matrix includes a selection of clinical units on a first axis, a selection of variables on a second axis, and a selection of metric functions on a third axis. The risk matrix may include a variable selector, a priority selector, or a risk threshold controller that the user can manipulate by operation of an input device, such as a mouse or pointer. The variable selector is operable by the user to configure the selection of variables to display in the three-dimensional matrix. The priority selector is operable by the user to select a priority level for variables displayed in the matrix. The risk threshold selector is operable by the user to select a current risk threshold for risk scores associated with the selection of metric functions, which further determines the amount of data displayed in the matrix. The variable selector, priority selector and risk threshold selector may (or may not) all be displayed on the same user interface screen at the same time, and may not always be displayed in the same order. In other words, in some embodiments, the variable selector may be displayed on a separate screen from the screen in which the priority level selector and risk threshold selector are displayed.

In one embodiment, the application program further includes program instructions that, when executed by the microprocessor, cause the microprocessor to automatically display a signal marker in the three-dimensional risk matrix to represent each combination of variable/unit/function of “v1”, “u1”, and “f1” for which “v1” is in the selection of variables, and “v1” has a variable priority greater than or equal to a variable priority selected by the priority selector, and “f1” is in the selection of functions, and risk score “S_{f1,v1}(u)” is greater than or equal to the current risk threshold (in embodiments where a higher risk score indicates higher risk and a lower risk score indicates lower risk) or the risk score “S_{f1,v1}(u)” is less than or equal to the current risk threshold (in embodiments where a lower risk score indicates higher risk and a higher risk score indicates lower risk). The programming instructions in the application program may be further configured to cause the microprocessor to automatically conceal from view (or refrain from displaying) every signal marker in the three-dimensional risk matrix that represents a combination of variable/unit/function of “v2”, “u2”, and “f2” for which “v2” is not in the selection of variables, or “v2” has a variable priority less than the variable priority selected by the priority selector, or “f2” is not in the selection of functions, or risk score “S_{f2,v2}(u2)” is less than the current risk threshold (in embodiments where a higher risk score indicates higher risk and a lower risk score indicates lower risk) or the risk score “S_{f1,v1}(u)” is greater than or equal to the current risk threshold (in embodiments where a lower risk score indicates higher risk and a higher risk score indicates lower risk). The application program further causes the microprocessor to dynamically repeat at least one of the automatically concealing and the automatically revealing steps when the user operates at least one of the variable selector, the priority selector, or the risk threshold selector in the user interface.

The application program includes programming instructions that cause the microprocessor to detect that the user has manipulated an input controller associated with the computer system to select a first signal marker displayed on the three-dimensional risk matrix displayed on the display device. When this happens, the application program is operable with the microprocessor to use the risk data object to identify a combination of variable/unit/function of “v3”, “u3”, and “f3” represented by the selected signal marker, and generate and display an investigation panel. The investigation panel prompts the user to select or confirm at least one of “v3”, “u3”, “f3”, a variable group associated with “v3”, or a plot type to use for root cause analysis of data represented by the selected signal marker. The system then generates and displays a grid including multiple plots rendered in accordance with the selected plot type.

In one embodiment, the plot type is a parallel coordinate plot, and the grid includes a combined plot for all clinical units combined, as well as individual plots for each clinical unit including “u3”. The combined plot illustrates metric values obtained by applying “f3” to the variables in the variable group in all clinical units combined, and each individual plot illustrates scaled metric values obtained by applying “f3” to the variables in the variable group only for one clinical unit. The metric values are scaled to allow simultaneous visualization of variable metrics that have varying values.

If the system detects that the user has manipulated the input controller to select a first profile marker in the combined plot, then the system visually highlights the first profile marker in the combined plot and automatically visually highlights a second profile marker on a second plot on the grid on the display device, where the first profile marker and the second profile marker are associated with the data of the same patient collected at the same clinical unit.

In some embodiments of the invention, the system also may be configured to display a subject data analysis controller and a dashboard communication controller on the grid. When the user manipulates the input device to activate the subject data analysis controller while a profile marker is highlighted on the grid, the system automatically generates and displays on the display device a subject data table including variables, measurements for the variables, and metric values for the measurements, for a patient associated with the highlighted profile marker. When the user manipulates the input device to activate the dashboard communication controller while the profile marker is highlighted on the grid in the user interface, the system automatically generates and displays on the display device a dashboard dialog panel configured to permit the user to create a risk alert record for the clinical unit and the variable associated with the highlighted profile marker. The risk alert record includes data fields for saving one or more of a clinical data unit identifier, a variable identifier, a metric for the variable, and a user-generated description of a risk. The system stores the risk alert record in the risk data object and transmits at least a portion of the risk data object to a remote issue tracking system for the clinical trial.

### BRIEF DESCRIPTION OF THE DRAWINGS

Embodiments of the present invention will hereinafter be described in detail below with reference to the accompanying drawings, wherein like reference numerals represent like elements. Where applicable, some features may not be illustrated to assist in the description of underlying features.

FIG. 1 shows a block diagram of a computer network configured to operate in accordance with certain embodiments of the present invention.

FIG. 2 illustrates an example overall data flow in embodiments of the present invention.

FIG. 3 illustrates an example clinical data set in embodiments of the present invention.

FIGS. 4 and 5 contain block diagrams that show two examples of computer networks where embodiments of the present invention could be used.

FIGS. 6-9 shows a flow diagram illustrating by way of example the steps performed by a clinical trial data analyzer in accordance with one form of the present invention.

FIGS. 10, 11A, 11B, 12A, 12B, 13A, and 13B show screen shots of an exemplary user interface screen for a clinical trial data analyzer configured to operate in accordance with certain embodiments of the present invention.

FIGS. 14 and 15 illustrate an exemplary flow diagram for an interactive user interface for a clinical trial data analyzer system configured to operate according to embodiments of the present invention.

FIGS. 16, 17, 18, 19, 20, 21, 22, 23, 24, 25, 26, 27, 28, 29, 30, 31, 32A, 32B, 33A, 33B, 34A, and 34B illustrate further exemplary screenshots for user interface for a clinical trial data analyzing system configured to operate according to embodiments of the present invention.

### DETAILED DESCRIPTION

Clinical trial data analysis typically involves analyzing data variables that have multiple dimensions. The multiple dimensions arise from the large multiplicity of patients, sites, variables, metric functions, etc. Without effectively reducing and/or otherwise managing such multiple dimensions, analysis of clinical trial data are bound to face challenges relating to speed, accuracy, and usability. However, features of the user interface provided by embodiments of the present invention reduce or eliminate problems associated with conventional clinical trial data analysis systems relating to speed, accuracy, and usability. For example, the three-dimensional risk matrix and the risk threshold controller provided by embodiments of the present invention are configured to selectively and dynamically display risk scores that indicate a risk associated with a certain clinical unit based on a metric function applied to measurements of certain variables, thereby reducing multiple dimension data to single dimension data for easier comparison and analysis.

In embodiments of the present invention, a user may slide the risk threshold controller up and down until the markers displayed in the three-dimensional risk matrix are dense enough and yet sparse enough to indicate a significant or interesting statistical anomaly or characteristic in the data. The identified anomaly/characteristic may be further investigated by selecting a marker and examining one of the multiple plots provided by the embodiments, each of which allowing for reduction in dimensionality and dynamically controlling opacity to isolate and/or emphasize the statistical anomaly or interesting characteristic. Further, the data objects generated and used by embodiments of the present invention are selected and arranged to be wholly self-contained, thereby eliminating the need to use more than one data object when a certain user interface feature is manipulated by a user and such manipulation requires the graphic representation of the data on the display device to be dynamically updated. Such configuration of the present data objects improves the speed in clinical data analysis and consequently also improves usability. Accordingly, embodiments of the present invention permit users to analyze clinical trial data in a faster, more efficient, and more useful manner.

Embodiments of the present invention use a cross-platform application software development framework, such as QT, and a data analytic engine such as R, SAS, C++ or any other appropriate software tool, to process and analyze clinical data, enrollment data, and adverse event data from clinical trials and present users with visualization tools that permit the users to selectively filter and display the data in accordance with risk levels associated with the values recorded for certain variables in the data. In one embodiment, data sets from a clinical trial (e.g., clinical data, enrollment data, and adverse event data) in .csv or other formats are imported through an import module. The import module recognizes and maps the variable names from each imported enrollment data set and each clinical data set in accordance with predefined labels used by the system. Alternatively, a user may perform the mapping. The user may also revise the mapping. Enrollment data set mapping may include mapping to a site, a geographic region, a trial participant, a treatment or intervention assigned, or a date of enrollment. Clinical data set mapping may include mapping to a site, a geographic region, a trial participant, a variable, a variable value, a visit, or a subvisit when subvisits are associated with a parent visit. Using an R data engine, the imported and mapped data are saved as a first object for later access. A known problem masker (or “shield”) may be activated if a user wishes to ignore or disregard certain measurements, certain variables, or certain sites in a particular session.

The first data object may be modified by the user to include additional attributes. The user may also modify the first data object by combining the data from multiple clinical sites or regions, renaming the combined sites and/or regions, grouping variables and giving a name to groups of variables, or by removing variables or sites, etc. The modified first data object may be viewed in the object viewer.

A user may then select metrics to operate on the data in the first data object. The metrics may include but are not limited to mean, median, standard deviation, repeated value frequency (across all values for a subject), carryover (frequency of exact values over contiguous visits), mean Euclidean distance between a value at one visit compared to the value of the immediately preceding visit, digit preference, etc. A metric calculation engine may calculate the metrics for the sites and variables for the data in the saved first object. The metrics together with at least a portion of the first data object may then be saved in a second data object.

The user may then select statistical tests to be executed on the metrics using the data in the second data object. These may include univariate testing using parametric, non-parametric, and Bayesian techniques, and/or multivariate testing including correlations, similarity, and cluster analysis. The statistical tests may generate risk scores for the sites, and may be saved in a third data object along with at least a portion of the second data object. Outcomes from the statistical testing stored in the third data object are passed to a visualization engine from which root-cause analysis can be performed. For example, the user may inspect a visualization and record results and actions in a dashboard, which may include saved snapshots of plots and graphs that reveal or indicate a presumed root cause. The dashboard may be saved and forwarded to other team members using user-preferred collaboration tools. The team members may investigate the findings and provide follow-up documentation of the results and, in a subsequent import of updated data, activate a shield to conceal, discard, or ignore data and/or findings that have already been inspected and/or analyzed in previous user sessions.

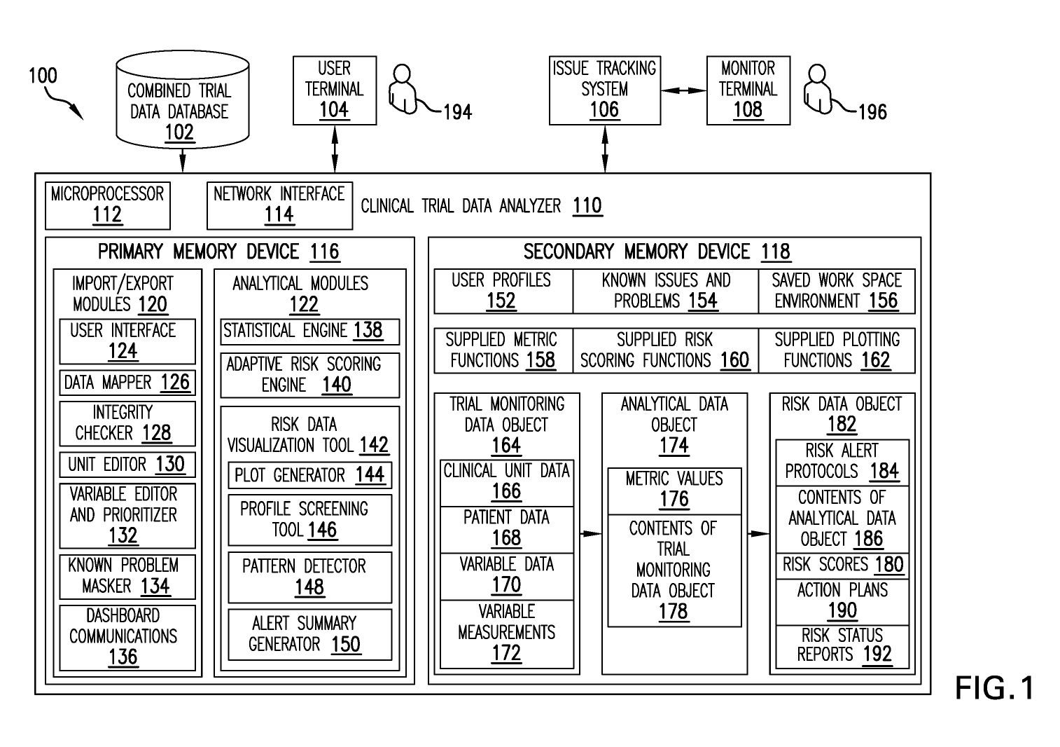

FIG. 1 shows a block diagram of a computer network 100 configured to operate in accordance with one embodiment of the present invention. As shown in FIG. 1, a clinical trial data analyzer 110 receives clinical trial data from a combined trial data database 102, and communicates with a user terminal 104 operable by a user 194 of the computer network 100 to import and analyze the clinical trial data stored in the combined trial data database 102. The clinical trial data analyzer 110 also communicates with an issue tracking system 106, which in turn communicates with a monitor terminal 108 used by another user 196 for monitoring issues identified by the clinical trial data analyzer 110 in the clinical trial data from combined clinical trial data database 102. The clinical trial data analyzer 110 may be connected to the combined trial data database 102, the user terminal 104 and the issue tracking system 106 via any suitable data communications channel or network, including without limitation the Internet (not shown in FIG. 1), a local area network (also not shown) or corporate intranet. In alternative embodiments, the combined trial data database 102, user terminal 104, issue tracking system 106 and monitor terminal 108 may all reside on, or be connected to, a single workstation or personal desktop computer, laptop computer, tablet computer or handheld computer system.

The clinical trial data analyzer 110 includes a microprocessor 112 (for executing various functions of the clinical trial data analyzer 110), a network interface 114 (for communicating with external devices and/or networks), a primary memory device 116 (such as random-access memory), and a secondary memory device 118, e.g., a hard disk drive. The primary memory device 116 stores one or more application programs in the form of executable program modules, including an import/export modules 120 for importing and exporting clinical trial data and metrics associated with the clinical trial data, as well as analytical modules 122 for performing statistical analysis on the clinical trial data stored in the combined trial data database 102. The analytical modules 122 also include a risk data visualization tool 142 for generating and displaying plots and graphic representations of the data and metrics associated therewith.

The import/export modules 120 include a user interface 124 for displaying information and prompts to a user and receiving corresponding user input and operating instructions. The import/export modules 120 also include a data mapper 126 configured to assist the user in mapping imported data to predefined variables recognizable by the clinical trial data analyzer 110. An integrity checker 128 verifies the integrity of the data as it is imported (to insure, for example, that all the data imported is in the proper format and comprises values that fall within a permissible range of values. For instance, the integrity checker 128 may be configured to display warnings or error messages to the user if an attempt is made to import heart rate measurements as fractions or alphabetic character strings instead of integers falling within the range of 25 and 250, or patient visit dates in the wrong format or impermissible date ranges. A unit editor 130 permits users to edit and/or correct the imported clinical unit data, if necessary, and a variable editor and prioritizer 132 permits users to edit and/or correct the values of imported patient variables, and assign priorities to each variable. The import/export modules 120 also includes a known problem masker 134, configured to permit the user to mask or filter data associated with known problems or issues in the data that are not considered by the user to be relevant or important for purposes of the current analytical session, and a communications dashboard 136 for interactive communications with the user.

The analytical modules 122 include a statistical engine 138 (for generating various statistical metrics based on variable measurements taken for a particular unit), an adaptive risk scoring engine 140 (for generating and assigning risk scores based on the generated metrics), and the risk data visualization tool 142 to generate and display visualizations of the assigned risk scores on a display device.

Adaptive Risk Scoring Engine

Preferred embodiments of the present invention feature an adaptive risk scoring engine 140 for generating and assigning risks scores to the metrics generated from the variable measurements for each clinical unit. Risk scores may be assigned using a variety of different methods and models. The adaptive risk scoring engine 140 comprises programming instructions that, when executed by the microprocessor, will cause the microprocessor to automatically select an optimal method and optimal model for assigning a risk score for each metric. The selection of a particular method or model depends on factors, including without limitation: the type of the metric (e.g., mean, variance, entropy, etc.), the type of variable (e.g., discrete, continuous), the amount of information in the given experimental unit (e.g. number variable values), the total amount of information across all units (i.e., the total number of variable values), the distribution of the given variable and its metric across the unit (e.g., normal, symmetric, skewed). The risk scoring engine first selects the testing framework (i.e., a single statistical test procedure vs a randomization test procedure), and then uses an adaptive model selection procedure to assign risk scores based on, but not limited to parametric, non-parametric, or Bayesian statistical testing frameworks by selecting an optimal approach to assign a risk score based on the factors listed above.

For instance, in one embodiment of the invention, the risk score assigned by the risk scoring engine 140 comprises a p-value, which is the probability of seeing a result from a comparison of the data at least as extreme as what was observed, assuming, for example, that there are no real differences between the comparators. In another embodiment, a Bayesian model is used to assign a risk score, where the risk scoring engine 140 may comprise programming instructions configured to cause the microprocessor to assign a risk score based on a posterior probability, where the posterior probability is defined as the probability of assumed parameters from a distribution given the data observed. In still another embodiment, the risk scoring engine 140 may be programmed to use a combination of two or more of the aforementioned methods, or to prompt the system user to select the most suitable method for the particular circumstances from a list of methods. In yet another embodiment, the risk scoring engine 140 may be configured to assign risk scores based on other testing. The following four examples illustrate four different ways of setting up the testing for assigning risk scores.

Example 1: Variable=systolic blood pressure (SBP). Metric=variance. Site X has at least ten patients and a total N patients>50. The test will be based on comparison of Site X data with total data from all sites (excluding the data at Site X). The parametric Inverse Chi Square model can be used to conduct the test.

Example 2: Variable=SBP. Metric=variance. Site X has five patients with a total N patients>50. The test will be based on a comparison of Site X data with total data for all sites (excluding the data at Site X). In this case, a randomization test utilizing t-test statistics may be employed. This procedure will produce 1000 realizations of the test statistics, where each test statistic is a result of a comparison between a randomly selected five patient subset verses the remaining data. Separately, the value of the test statistics will be calculated for the five patients coming from Site X (T5) and compared with the distribution of 1000 test statistics described above. This comparison will result in a risk score, which would be the probability of observing T5 at random.

Example 3: Variable=SBP. Metric=carry over rate. Site X has 15 patients with total N patients>70. The test will be based on a comparison of Site X data with total data for all sites (excluding the data at Site X). In this case, a beta distributional model could be used to conduct the test.

Example 4: Variable=SBP. Metric=entropy. Site X has 15 patients with total N patients>70. In this case, the test might be based on a comparison of Site X data with total data (excluding the data from site X). A gaussian distributional model could be used to conduct the test.

UBER RISK SCORE: From these individual risk scores an Uber risk score can be constructed by the user, who assigns weights to each occurrence of a risk score beyond the threshold for the metric and variable, thereby creating a unit ranking and identifying the units with the highest risk across all variables and metrics of interest. The Uber risk score may then be used by the risk data visualization tool 142 to generate and display three- and four-dimensional risk matrices on display devices associated with the clinical trial data analyzer 116.

In addition to the aforementioned examples, it will be recognized and appreciated by those skilled in the art that there are many other possible ways to assign risk scores (or program a computer system to assign risk scores) for a collection of metrics associated with a clinical unit without departing from the scope of the claimed invention.

In preferred embodiments, the risk data visualization tool 142 comprises a plot generator 144 for presenting various plots to a user based on the clinical data and the output of the statistical engine, a profile screening tool 146 for selectively highlighting and profiling data associated with a particular trial participant, a pattern detector 148 for automatically detecting predefined patterns in parallel coordinate and density plots, and an alert summary generator 150, which assists a user in creating an alert when an issue is identified.

The secondary memory device 118, which may comprise, for example, a persistent memory device, such as a hard disk drive attached to a personal computer, stores user profiles 152, known issues and problems 154, saved workspace environment details 156, supplied metric functions 158 (for generating various statistical metrics based on measured patient variables), supplied risk scoring functions 160 (for generating various risk scores based on the generated metrics), and supplied plotting functions 162 (for generating various plots based on any combination of units, variables, metric functions, and risk scores). The secondary memory device 118 further stores three data structures, including a trial monitoring data object 164, an analytical data object 174, and a risk data object 182. As will be described in more detail below, at least a portion of the trial monitoring data object 164 is replicated in the analytical data object 174, and at least a portion of the analytical data object 174 is replicated in the risk data object 182 so that the clinical trial data analyzer 110 only needs to reference a single self-contained data structure in memory at any given time to carry out any of the interactive statistical analysis or visualization functions performed by the clinical trial data analyzer 110.

The trial monitoring data object 164 is a data structure stored on the secondary memory device 118 that holds clinical trial data imported into the clinical trial data analyzer 110 from the combined trial data database 102. The imported clinical trial data stored in the trial monitoring data object 164 typically comprises at least clinical unit data 166, patient data 168, variable data 170, and variable measurements 172. Statistical functions are performed on the data in the trial monitoring data object 164 to generate metric values 176 associated with the patient data 168 and variable measurements 172 stored in the trial monitoring data object 164, which are then stored in the analytical data object 174. As previously stated, the analytical data object 174 also includes some or all the contents of the trial monitoring data object 178 so that the analytical data object 174 alone may be used by the clinical data analyzer 164 to support any functionality related to analyzing or visualizing the clinical trial data, without it being necessary to retrieve or access the trial monitoring data object 164. As will be described in more detail below, the analytical data object 174 may be used to generate a risk data object 182 that includes risk alert protocols 184, assigned risk scores 180, action plans 190, and risk status reports 192, as well as some or all of the contents of the analytical data object 186 so that the risk data object 182 is fully self-contained and therefore can support any functionality related to interactive statistical analysis or visualization of the clinical trial data without it being necessary to retrieve and access either the analytical data object 174 or the trial monitoring data object 164 on the secondary memory device 118. The risk data object may also include risk summaries (not shown) comprising user-generated descriptions and documentation about identified risks and assigned risk scores.

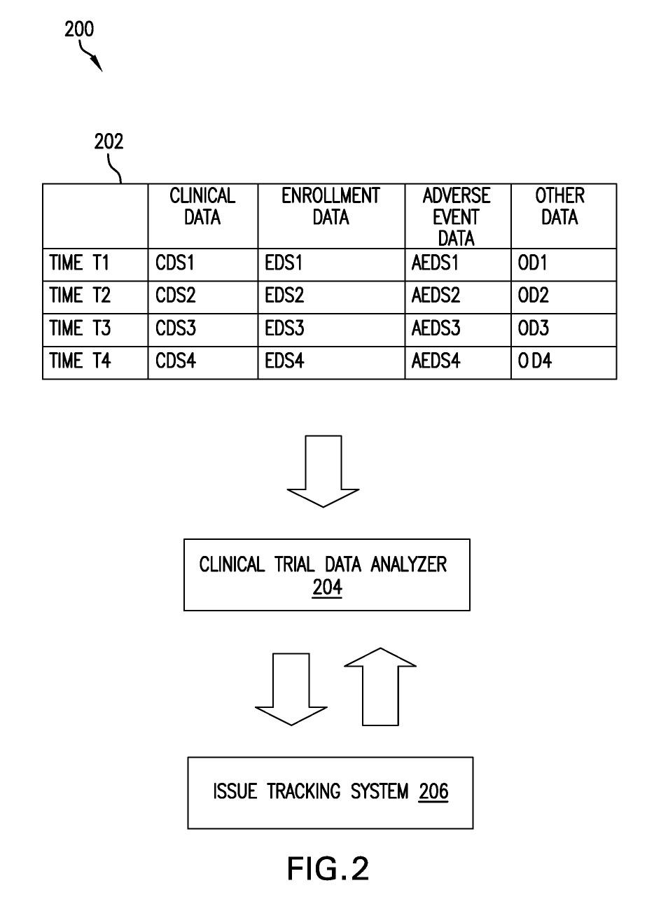

FIG. 2 shows a diagram illustrating by way of an example an overall data flow 200 in embodiments of the present invention. Clinical trial data sets 202, which may include clinical data, enrollment data, adverse event data and other data, may be imported into a clinical data analyzer 204. The clinical trial data analyzer 204 uses the clinical trial data sets 202 to assign risk scores to certain patient variable measurements and generate risk alerts and risk summaries. The assigned risk scores, risk alerts and risk summaries are then exported to an issue tracking system 206. The clinical data analyzer 204 may also be configured to receive and import corresponding status reports from the issue tracking system 206. Such status reports will typically provide the clinical data analyzer (and its users) with current information about the handling and status of a particular data anomaly or issue.

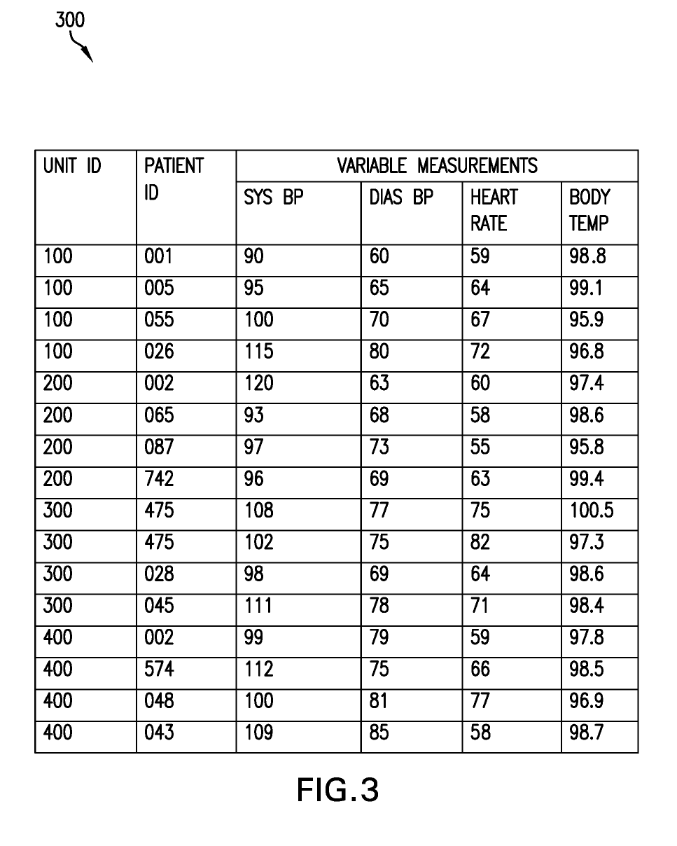

FIG. 3 illustrates an example of a clinical trial data set 300 used in some embodiments of the present invention. A clinical trial data set 300 may include multiple records. Each record includes fields storing a unit identifier, a patient identifier, and a few of the variables associated with the patients at the clinical unit. In this example, the variables include the patient's systolic and diastolic blood pressure, heart rate, and body temperature. Below the variables are the variable measurements. For example, the measurements for patient number 005 for the variables systolic blood pressure, diastolic blood pressure, heart rate, and body temperature are 95, 65, 64, and 99.1, respectively.

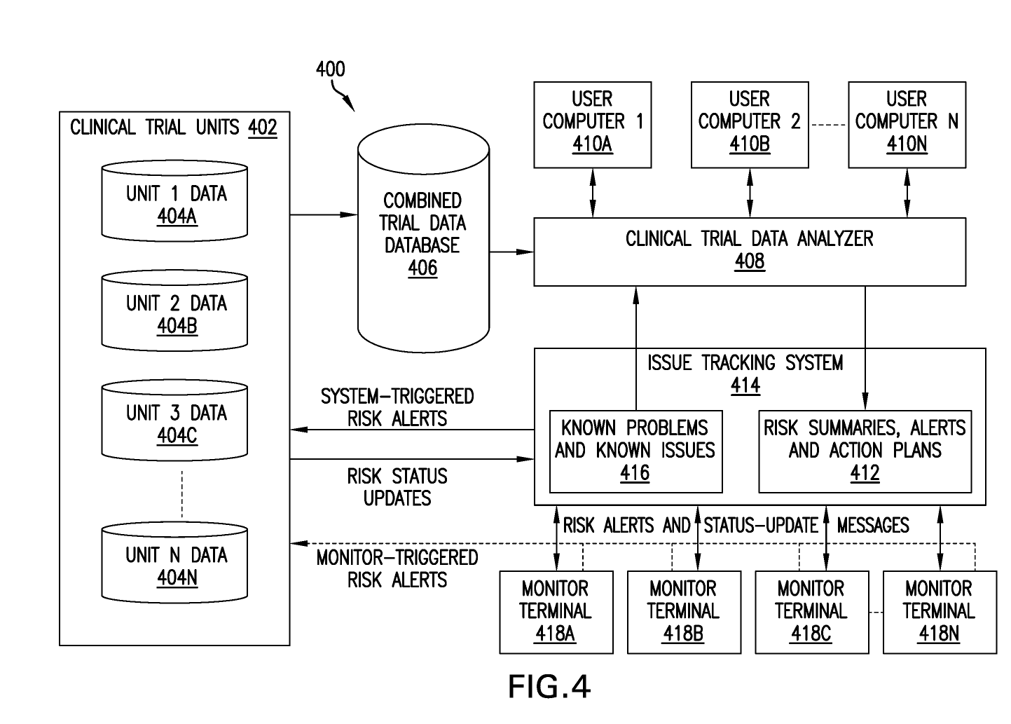

FIG. 4 shows an example block diagram of a network architecture 400 in certain embodiments of the present invention. As shown in FIG. 4, a number of clinical trial units 402 may participate in a clinical trial. During the clinical trial, the clinical trial units 402 record unit, patient, variable and measurement data for their respective units to produce a collection of individual clinical trial unit data sets 404A-404N. Data from multiple data sets 404A-404N is sent to, and stored in a combined trial data database 406. A clinical data analyzer 408 reads the data from the combined trial data database 406 communicates with various user computers 410A-410N to assist users in analyzing the data from the combined trial data database 406 to generate metrics and metric signals for each unit, patient, variable and measurement in the data contained in the combined trial data database 406, and assign risk scores to each signal. The clinical data analyzer 408 also assists users by displaying graphic representations of the metrics, measurements and signals on display devices associated with the user computers 410A-410N. Based on the results of the analysis, the clinical data analyzer 408 may be configured to send risk summaries, alerts, and action plans 412 to an issue tracking system 414, and receive known problems and known issue information 416 from the issue tracking system 414.

The issue tracking system 414 may be configured to automatically generate and send system-triggered risk alerts to the clinical trial units 402, where those system-triggered alerts may be received and acted upon, if necessary, by clinical trial participants responsible for collecting and recording clinical trial data and/or implementing data collection practices intended to reduce errors and guarantee compliance. Typically, the issue tracking system 414 is further configured to periodically request and receive risk status updates from the clinical trial participants so that the issue tracking system 414 will have a record of actions taken and current statuses for each system-triggered risk alert generated and transmitted to the clinical trial units 402 by the issue tracking system 414. In some embodiments, instead of (or in addition to) automatically generating and sending system-triggered risk alerts directly to the clinical trial units 402, the issue tracking system 414 may be further configured to transmit risk summaries, alerts and/or action plans to one or more monitor terminals 418A-418N operated by human clinical trial monitors (not shown in FIG. 4) who are responsible for monitoring and overseeing data collection and recording practices at the clinical trial units 402. The human clinical trial monitors may then generate monitor-triggered risk alerts and action plans, which may be delivered to the clinical trial units 402 by the human clinical trial monitors by electronic means or by visiting the clinical trial units 402 in person, if appropriate, to ensure that any compliance problems indicated by the risk summaries, alerts and action plans are corrected. The monitor terminals 418A-418N operated by the human clinical trial monitors also may be configured to receive and collect risk status updates from the clinical trial units 402 and transmit those risk status updates to the issue tracking system 414.

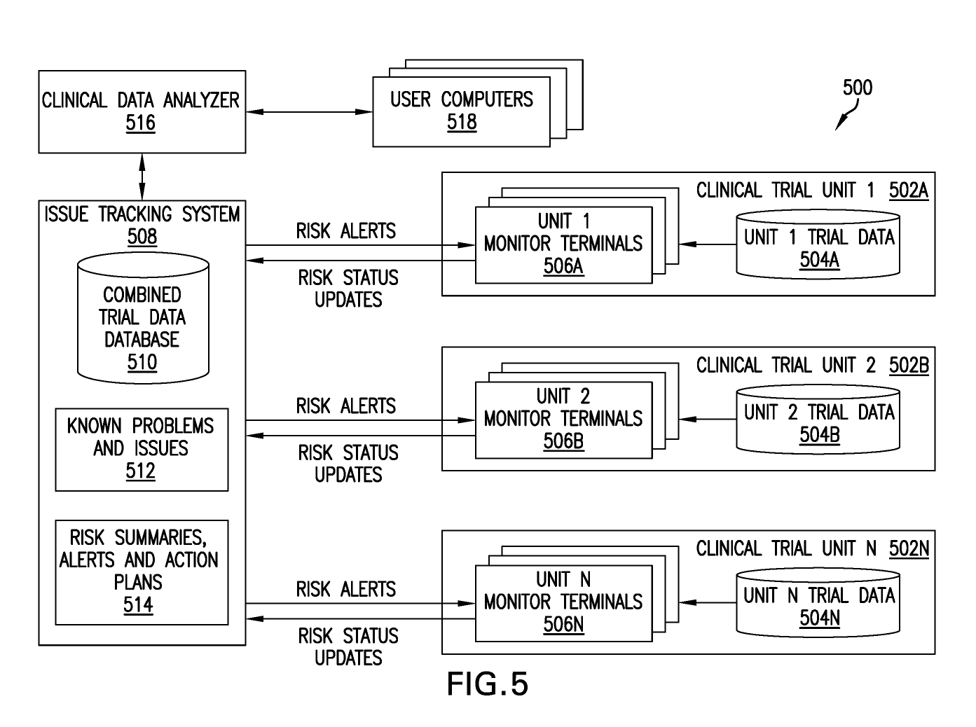

FIG. 5 illustrates an example block diagram of an alternative network architecture 500 in another embodiment of the present invention. In this embodiment, each clinical trial unit 502A-502N includes a trial data set 504A-504N and monitor terminals 506A-506N, respectively. Trial data stored in the trial data sets 504A-504N may be retrieved and displayed on the monitor terminals 506A-506N at each clinical trial unit 502A-502N. The monitor terminals 506A-506N for the clinical trial units 502A-502N communicate through a data communications network (not shown) with an issue tracking system 508 to receive risk alerts and provide risk status updates associated with the trial data by operation of a clinical trial data analyzer 516. The issue tracking system 508 includes a combined trial data database 510 that stores combined trial data from various clinical trial units 502A-502N. The issue tracking system 508 also stores known problems and issues 512 as well as risk summaries, alerts, and action plans 514. The issue tracking system 508 communicates with the clinical data analyzer 516, which is in turn communicatively connected to various user computers 518. The clinical trial data analyzer 516 applies statistical functions to the clinical trial data to produce metrics for all the measurements in the trial data and assigns risk scores to those metrics and measurements according to the algorithms described herein.

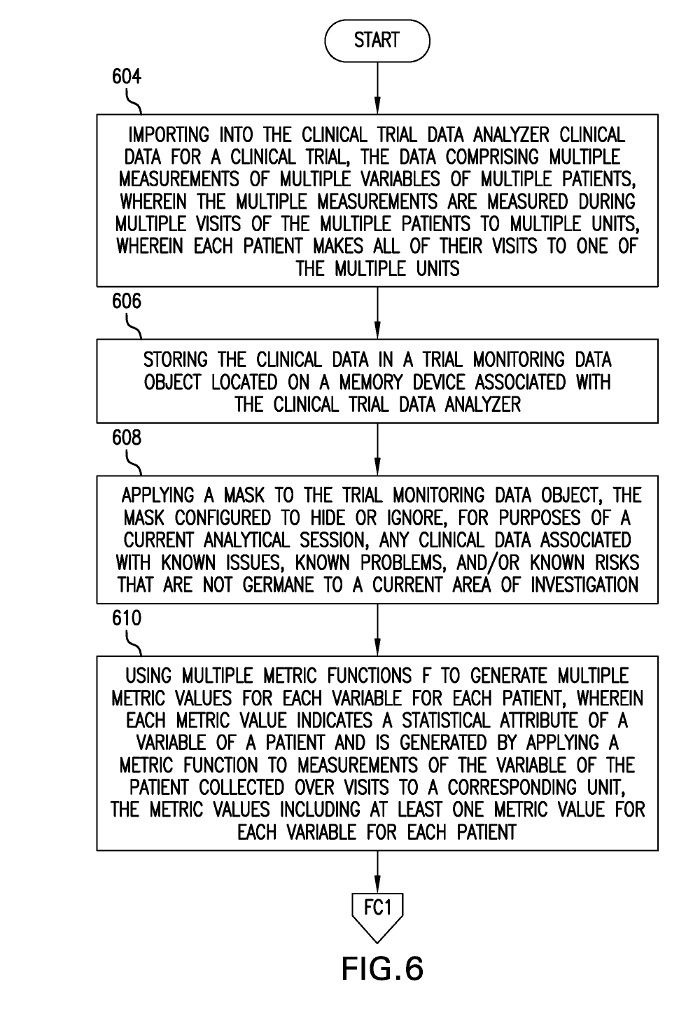

FIGS. 6-9 show a high-level flow diagram illustrating by way of example the steps carried out by a clinical trial data analyzer configured to execute algorithms in accordance with forms of the present invention to analyze clinical data from a clinical trial. As stated above, clinical trial data may comprise clinical data, enrollment data, adverse event data, or any other type of data generated for a clinical trial or investigation program for a pharmaceutical product, medical treatment or device for human patients. Although the flow diagram shown in FIGS. 6-9 illustrate the steps performed to process and assign risk scores to clinical data, it will be understood and appreciated by those skilled in the art that algorithms and processes described herein may also be beneficially used to process and assign risk scores to other types of clinical trial data, including without limitation, enrollment data and adverse event data.

As shown in FIG. 6, the process begins at step 604, where clinical data collected during a clinical trial are imported into the clinical trial data analyzer. The data may include multiple measurements for multiple variables (e.g., systolic and diastolic blood pressure, temperature, heart rate, height, weights, tumor size, etc.) for multiple patients. Measurements for each patient are obtained during multiple visits of the patient to a clinical trial unit (e.g., a health provider site, a geographic region such as a city or a country, etc.) participating in the clinical trial. In one embodiment, each patient visits a single clinical unit multiple times. That is, data related to each patient are associated with only one of the clinical units among multiple clinical units participating in the clinical trial. It will be understood and recognized by those skilled in the art, however, that the system and process described and claimed herein may be modified, as appropriate, to account for situations in which one or more patients visit multiple clinical units over time, without departing from the scope of the claimed invention.

At step 606, the clinical trial data are stored in a trial monitoring data object located on a memory device associated with the clinical trial data analyzer. In one embodiment, a mask may optionally be applied to the trial monitoring data object at step 608 to hide or ignore, for purposes of a current analytical session, any clinical data associated with known issues, known problems, and/or known risks that are not germane to a current area of investigation. For example, if a clinical unit has been previously flagged as providing unreliable blood pressure data, the blood pressure measurements from the clinical unit (or all clinical units) may be masked out (or ignored) in the current analytical session to permit the user to more easily focus on other variables and other measurements for that clinical unit without the analysis being burdened, unduly distracted or influenced by the known unreliable blood pressure measurements.

At step 610, multiple metric functions are applied to the clinical data measurements to generate multiple metric values for each variable for each patient. A metric value is generated by applying a metric function to multiple measurements of a variable of a patient collected over multiple visits to a clinical unit. The metric function may be any statistical function considered to be appropriate and/or useful for indicating a statistical attribute for the variables, such as standard deviation, entropy, mean value, average value, rate of identical measurements, the sum of distances from neighboring visits (visits immediately before and after), the occurrence of similar within tolerance measurements, etc. For instance, if the metric function to be applied is the standard deviation and the variable is a heart rate of a patient, then the metric value is the standard deviation across the entire set of heart rate measurements for the patient recorded over multiple visits by that patient to a clinical unit. In one embodiment, at least one metric value is generated for each variable for each patient.

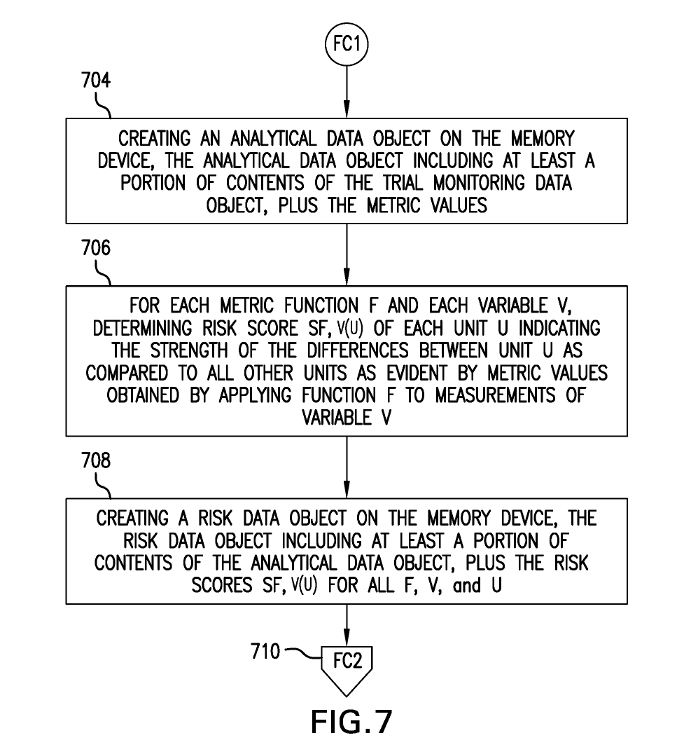

After generating the metric values at step 610, processing then continues at step 704 in FIG. 7 by way of flow chart connector FC1. In step 704, an analytical data object is created on the memory device to store the metric values, as well as at least a portion of the contents of the trial monitoring object (e.g. clinical unit information, variable information, etc.). Such data portion is replicated so that the analytical data object is wholly self-contained, and thereby capable of fully supporting further statistical clinical data analysis and visualization steps, as described in more below, without needing to retrieve or access the original trial monitoring data object. That is, in the following description, the use of the analytical data object for performing a certain step does not necessitate the use of any other data object besides the analytical data object.

Next, at step 706, for each metric function “f” and each variable “v”, a risk score Sf,v(u) is determined and assigned for each clinical unit “u.” As previously stated, there are a variety of different methods and techniques known in the art for determining and assigning suitable risk scores for a collection of metrics. The risk score Sf,v(u) indicates a relative strength of the differences between unit “u” as compared to all other units, in light of the metric values obtained by applying “f” to measurements of “v”. For example, in one embodiment, if the metric function is the standard deviation “sd” and the variable is the systolic blood pressure “sbp” of the patient, then the metric value “MVsd,sbp(pat)” of the patient “pat” is the standard deviation of blood pressure measurements of the patient collected over multiple visits to a clinical unit “u”. The corresponding risk score Ssd,sbp(u) indicates how different is the collection of “MVsd,sbp(pat1), MVsd,sbp(pat2), MVsd,sbp(pat3), . . . ” of patients “pat1”, “pat2”, “pat3”, . . . that visited unit “u”, as compared to the collection of “MVsd,sbp(patx1), MVsd,sbp(patx2), MVsd,sbp(patx3), . . . ” of patients “patx1”, “patx2”, “patx3”, . . . that visited all other clinical units combined.

The risk scores may be obtained by applying a scoring function to “MVsd,sbp(pat1), MVsd,sbp(pat2), MVsd,sbp(pat3), . . . , MVsd,sbp(patx1), MVsd,sbp(patx2), MVsd,sbp(patx3), . . . ” In some embodiments, the higher the risk score, the more different a clinical unit is from the rest of the clinical units. For example, the higher Ssd,sbp(u) is, the more different “u” is from the other clinical units as indicated by metric values “MVsd,sbp(pat)” of all patients of all units. In other embodiments, the lower the risk score, the more different a clinical unit is from the rest of the clinical units. For example, the lower Ssd,sbp(u) is, the more different “u” is from the other clinical units as indicated by metric values “MVsd,sbp(pat)” of all patients of all units.

The value of “Sf,v(u)” indicates the likelihood of an error in measurements of variable “v” at unit “u” given measurements of same or related variables at other units. For example, “Ssd,sbp(u)” indicates the likelihood of an error in measurements of systolic blood pressure at clinical unit “u” given measurements of systolic blood pressure or other related variables (e.g., diastolic blood pressure, heart rate, etc.) at other clinical units.

The risk scoring function may generate and assign the risk score “Ssd,sbp(u)” based on a comparison of the collection of “MVsd,sbp(pat1), MVsd,sbp(pat2), MVsd,sbp(pat3), . . . ” with the collection of “MVsd,sbp(patx1), MVsd,sbp(patx2), MVsd,sbp(patx3), . . . ” In one embodiment, “Ssd,sbp(u)” measures the magnitude of a probabilistic difference between the collection of “MVsd,sbp(pat1), MVsd,sbp(pat2), MVsd,sbp(pat3), . . . ” and the collection of “MVsd,sbp(patx1), MVsd,sbp(patx2), MVsd,sbp(patx3), . . . ”

For example, the risk scoring function may include hypothesis testing. The null hypothesis “HOsd,sbp(u)” is that the probabilistic distribution of the collection of “MVsd,sbp(pat1), MVsd,sbp(pat2), MVsd,sbp(pat3), . . . ” is not different, beyond a given threshold, than the probabilistic distribution of the collection of “MVsd,sbp(patx1), MVsd,sbp(patx2), MVsd,sbp(patx3), . . . ” In this embodiment, “Ssd,sbp(u)” is generated based on the difference between the probabilistic distribution of the collection of “MVsd,sbp(pat1), MVsd,sbp(pat2), MVsd,sbp(pat3), . . . ” and the probabilistic distribution of the collection of “MVsd,sbp(patx1), MVsd,sbp(patx2), MVsd,sbp(patx3), . . . ”

The hypothesis testing may comprise any applicable hypothesis testing known in the art. For example, the hypothesis testing may use a parametric or nonparametric framework that depends on the type of the variable or the type of the metric function. For instance, in some embodiments, the parametric model may be binomial when the metric function is a rate of occurrence, or may be Chi-square when the metric function is standard deviation. In another embodiment, the risk score Sf,v(u) may comprise a P-value from a statistical test, where the P-value is the probability of observing metric values at least as extreme as metric values obtained by applying “f” to measurements of “v” at “u”, assuming that there are no differences between “u” and other clinical units. In another embodiment, the risk score, SF,v(u), may use a Baysian framework to assign a posterior probability as the value of the risk score.

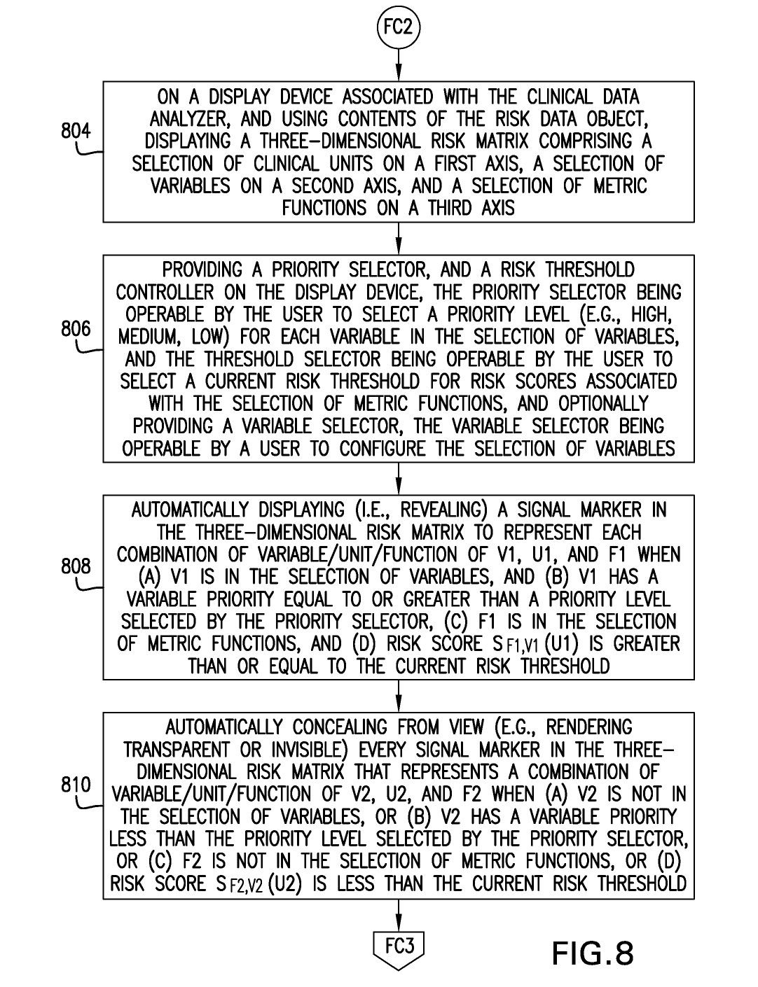

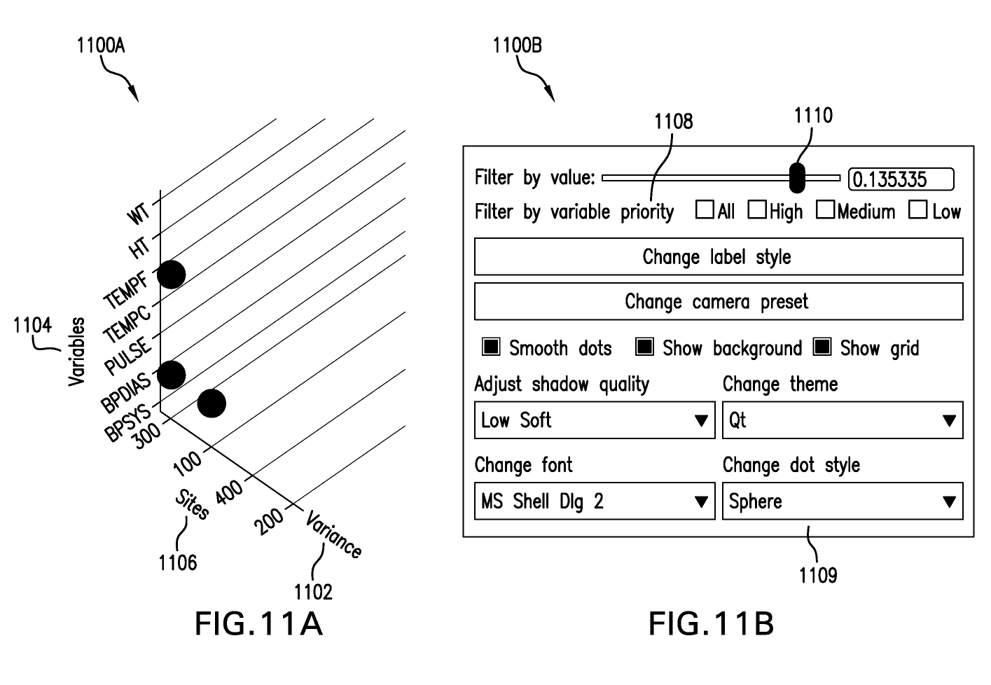

At step 708 a risk data object is created on the memory device. The risk data object includes risk scores “Sf,v(u)” for all metric functions “f”, variables “v”, and units “u”, as well as at least a portion of the contents of the analytical data object (e.g. clinical unit information, variable information, etc.). Such data portion is replicated so that the risk data object is wholly self-contained, and therefore capable of supporting statistical clinical data analysis as described below without relying on any other data object. After the risk scores are assigned, processing continues at step 804 of FIG. 8 by way of flow chart connector FC2, where, using the contents of the risk data object, a three-dimensional risk matrix is displayed on a display device associated with the clinical data analyzer. The three-dimensional risk matrix (depicted in FIG. 11A and described in more detail below) includes a selection of clinical units (or all clinical units) on a first axis, a selection of variables on a second axis, and a selection of metric functions on a third axis. In one embodiment, the selection of variables includes a group of related variables (e.g., systolic blood pressure, diastolic blood pressure, heart rate, and body temperature). Next, as shown at step 806 of FIG. 8, the system provides a priority selector and a risk threshold controller on the display device. The priority selector is operable by the user, in some embodiments, to select a priority level (e.g., high, medium, low) for the variables shown on the three-dimensional matrix. The risk threshold selector is operable by the user to select a current risk threshold for risk scores associated with the selection of metric functions.

In some embodiments, as shown in step 806 of FIG. 8, the system may also be configured to display on the display device a variable selector, which is operable by a user to configure the selection of variables for the matrix, for example, to remove a variable from the selection, to add a variable to the selection, or to change the order of the selected variables. The variable selector used to select the variables, or to select a group of variables, may be presented on the display device with the risk matrix, or alternatively before or after the risk matrix is displayed on the display device. In some embodiments, for example, the variable selector may be used to select variables (or a group of variables) during the step of importing the clinical data into the trial data object, in order to reduce the amount of data that has to be imported. In other embodiments, the variable selector may not be presented on the display device until after the user has been shown a parallel plot or density plot, whereupon the user may then use the variable selector to select a particular variable (or group of variables) to add to or remove from the plot. The variable selector, the priority selector, and the risk threshold selector may, or may not, appear together on a single screen or dialog.

The system is configured to automatically reveal or conceal signal markers in accordance with the operation of the variable selector, the priority selector and the risk threshold selector. The revealing step may include, at step 808, automatically displaying (or revealing) a signal marker (such as a color-coded ball) in the three-dimensional risk matrix to represent each combination of variable/unit/function of “v1”, “u1”, and “f1” for which “v1” is in the selection of variables, and “v1” has a variable priority greater than or equal to a priority level selected by the priority selector, and “f1” is in the selection of metric functions, and risk score “Sf1,v1(u)” is greater than or equal to the current risk threshold (in embodiments where a higher risk score indicates higher risk and a lower risk score indicates lower risk) or the risk score “S_{f1,v1}(u)” is less than or equal to the current risk threshold (in embodiments where a lower risk score indicates higher risk and a higher risk score indicates lower risk). The concealing step may include, at step 810, automatically concealing from view (rendering transparent or invisible) every signal marker in the three-dimensional risk matrix that represents a combination of variable/unit/function of “v2”, “u2”, and “f2” for which “v2” is not in the selection of variables, or “v2” has a variable priority less than the variable priority selected by the priority selector, or “f2” is not in the selection of metric functions, or risk score “Sf2,v2(u2)” is less than the current risk threshold.

In some embodiments, metrics with higher numerical risk scores will be considered to have a higher risk of being erroneous. In other embodiments, higher numerical risk scores may be indicative of a lower risk of error. If higher numerical risk scores are associated with a lower risk of error, and lower numerical risk scores are associated with (or indicative of) a higher risk of error, then the revealing step may include automatically displaying (or revealing) a signal marker (such as a color-coded ball) in the three-dimensional risk matrix to represent each combination of variable/unit/function of “v1”, “u1”, and “f1” for which “v1” is in the selection of variables, and “v2” has a variable priority greater than or equal to a priority level selected by the priority selector, and “f1” is in the selection of metric functions, and risk score “Sf1,v1(u)” is less than or equal to the current risk threshold, and the concealing step may include automatically concealing from view (rendering transparent or invisible) every signal marker in the three-dimensional risk matrix that represents a combination of variable/unit/function of “v2”, “u2”, and “f2” for which “v2” is not in the selection of variables, or “v2” has a variable priority less than the priority level selected by the priority selector, or “f2” is not in the selection of metric functions, or risk score “Sf2,v2(u2)” that is greater than the current risk threshold.

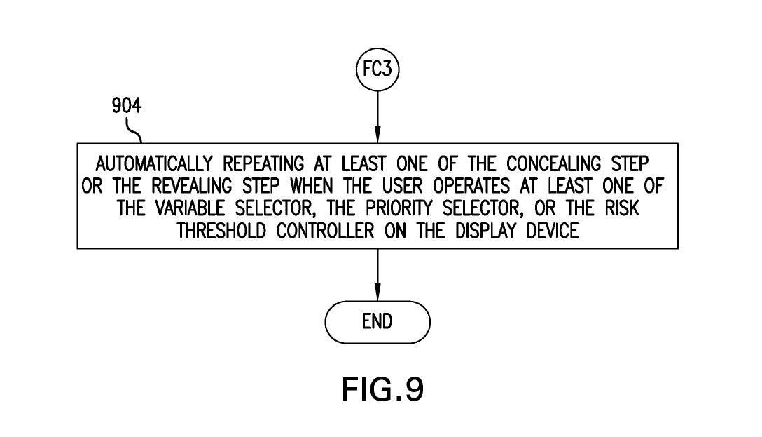

Processing then continues at step 904 in FIG. 9 by way of flow chart connector FC3. As shown in FIG. 9 at step 904, at least one of the concealing and revealing steps are automatically repeated when the user operates at least one of the variable selector, the priority selector, or the risk threshold selector on the display device. For example, one or more additional signal markers may be automatically revealed on the three-dimensional risk matrix by repeating the revealing step when the user operates the risk threshold controller to select a lower risk threshold, and one or more signal markers may be concealed/removed from the three-dimensional risk matrix by repeating the concealing step when the user operates the risk threshold controller to select a higher risk threshold. After performing step 904, process ends.

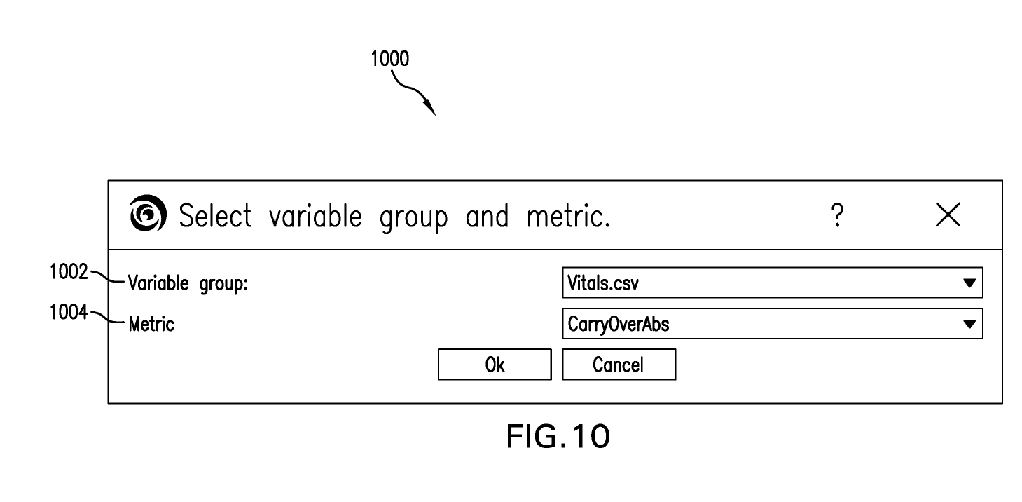

FIGS. 10, 11A, 11B, 12A, 12B, 13A, and 13B illustrate exemplary user interface screens (i.e., screenshots 1000, 1100A, 1100B, 1200A, 1200B, 1300A, and 1300B, respectively) generated and displayed by the risk data visualization tool 142 to assist the user in interactively analyzing and detecting patterns in the risk scores assigned to the metrics. As shown in the screenshot 1000 of FIG. 10, when the risk data visualization tool 142 is activated, the user may be presented with a screen for selecting a variable group 1002 and metric 1004 for generating a three-dimensional risk matrix. The screenshot 1100A in FIG. 11A illustrates an example of the resulting three-dimensional risk matrix when the user selects the metric to be variance 1102 as indicated on one axis of the matrix. A second axis indicates the selected variables 1104, which, in this case, are the variables weight, height, temperature, pulse, diastolic blood pressure, and systolic blood pressure. A third axis indicates the clinical units 1106. In this case, the clinical units 1106 are clinical sites 300, 100, 400, and 200.

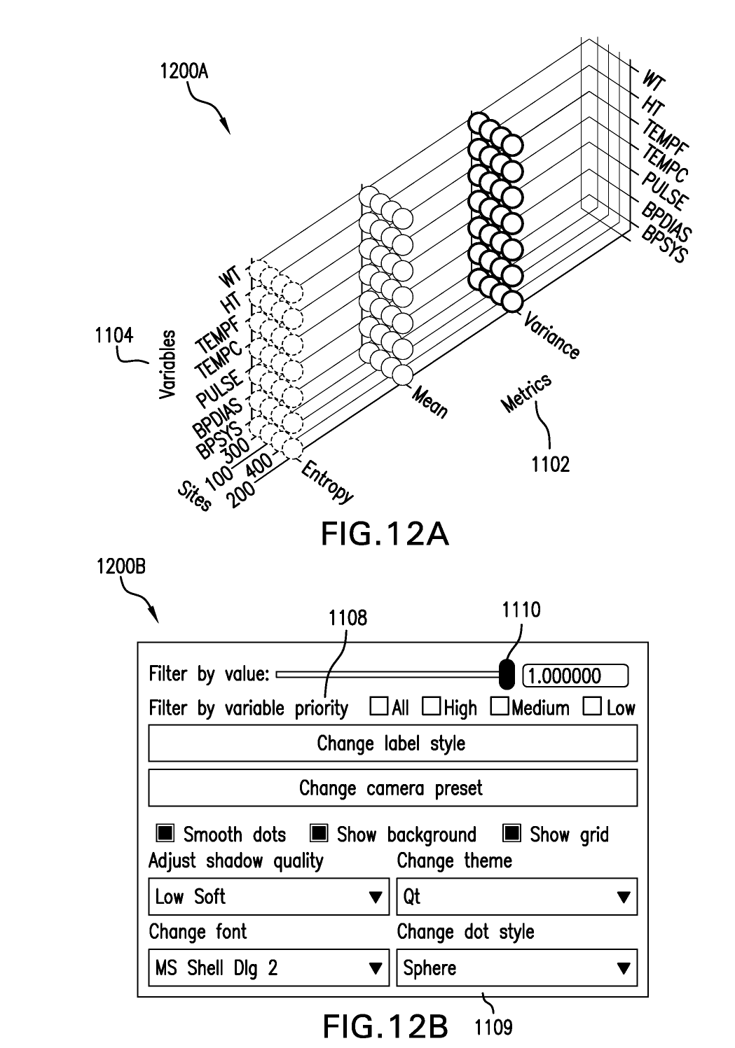

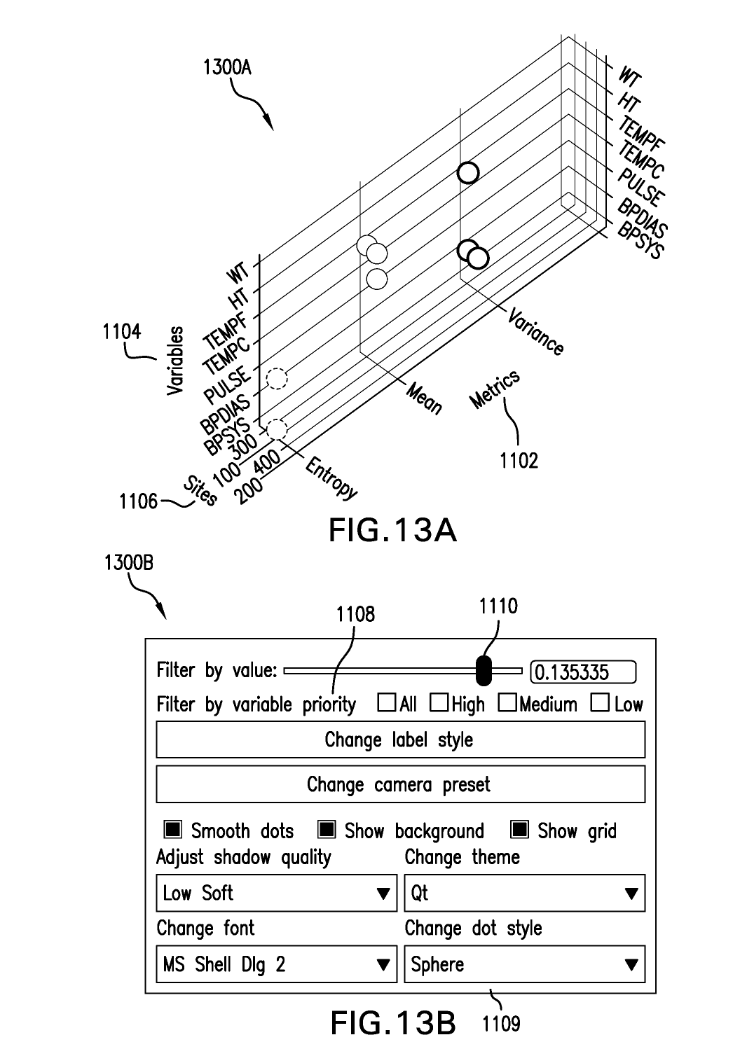

The screenshot 1100B in FIG. 11B illustrates an example of dialog box 1109 presented by the risk visualization tool 142, the dialog box 1109 comprising a priority selector 1108 and a risk threshold controller 1110. This dialog box 1109 may be displayed next to the three-dimensional risk matrix shown in the screenshot 1100A in FIG. 11A. The screenshots 1200A and 1200B in FIGS. 12A and 12B, respectively, show the same risk matrix and dialog box 1109 containing the priority selector 1108 and the risk threshold controller 1110 when the selected metrics 1102 are entropy, mean, and variance, and the risk threshold controller 1110 is moved all the way to the far right on the slider so that all of the risk scores pass the threshold test and are therefore revealed (i.e., visualized as respective “balls” in the matrix). Screenshots 1300A and 1300B in FIGS. 13A and 13B respectively show the same risk matrix and dialog box 1109 containing the priority selector 1108 and the risk threshold controller 1110 when the risk threshold is changed (by moving the risk threshold controller 1110 toward the left on the slider, i.e., away from its maximum value on the far right) so that fewer risk scores pass the threshold test and are revealed, and the rest are concealed.

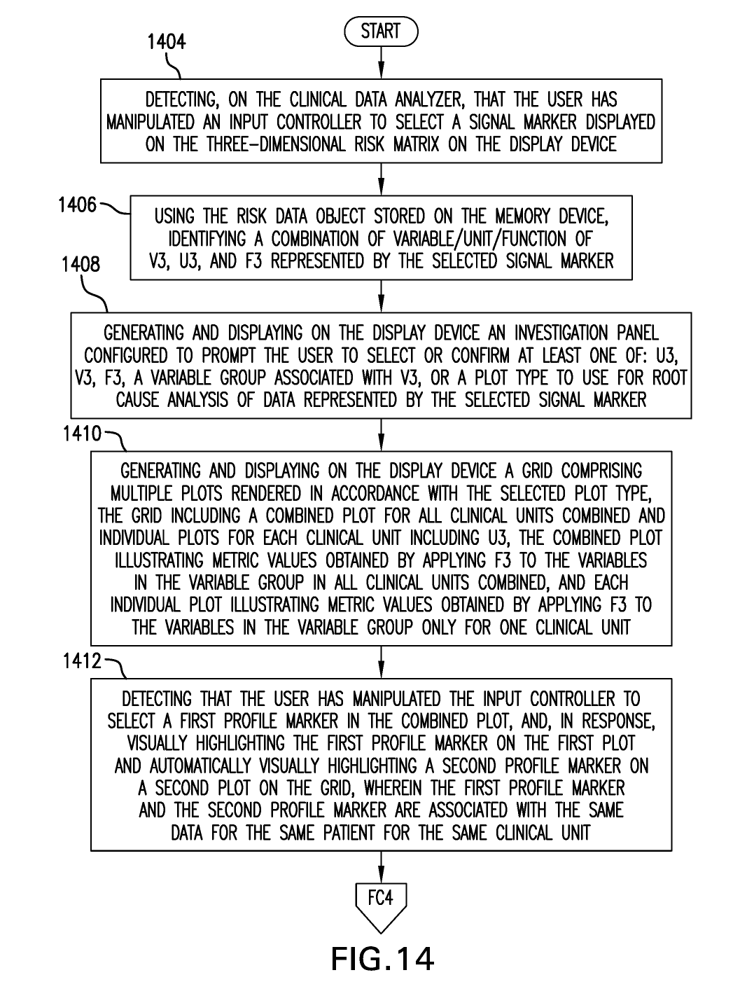

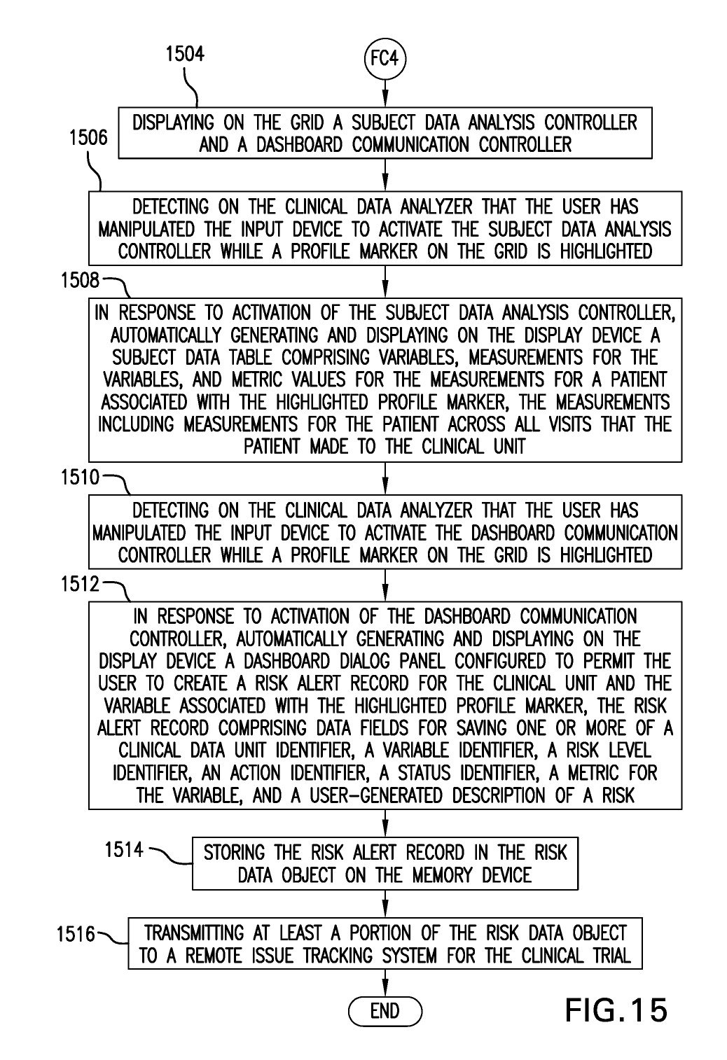

FIGS. 14 and 15 show a flow diagram illustrating by way of example the steps performed by the system to assist the user in further analyzing the data displayed on the three-dimensional risk matrix, such as the three-dimensional risk matrix shown in FIG. 13A. As shown in FIG. 14, the process begins at step 1404, where the risk data visualization tool 142 of the clinical trial data analyzer 110 detects that the user has manipulated an input controller to select a signal marker (e.g., a color-coded ball or other icon) displayed on the three-dimensional risk matrix on the display device. The risk data object 182 stored on the secondary memory device 118 is used (at step 1406) to identify a combination of variable/unit/function of “v3”, “u3”, and “f3” represented by the selected signal marker. At step 1408, an investigation panel is generated and displayed on the display device. The investigation panel is configured to prompt the user to select or confirm at least one of “v3”, “u3”, “f3”, a variable group associated with “v3”, or a plot type to use for root cause analysis of data represented by the selected signal marker. At step 1410, the risk data visualization tool 142 generates and displays a grid comprising multiple plots rendered in accordance with the selected plot type. In one embodiment, the grid includes a plot for all clinical units combined, as well as individual plots for each clinical unit, including clinical unit “u3”. The combined plot illustrates metric values obtained by applying the function “f3” to the variables in the variable group in all clinical units combined, and each individual plot shows metric values obtained by applying the function “f3” to the variables in the variable group only for one clinical unit. At step 1412, the risk data visualization tool 142 detects that the user has manipulated the input controller to select a first profile marker in the combined plot, and in response, the risk data visualization tool 142 visually highlights the first profile marker in the combined plot. The risk data visualization tool 142 also automatically visually highlights a second profile marker on a second plot on the grid, where the first and second profile markers are associated with the data of the same patient collected at the same clinical unit.

Processing next continues at step 1504 of FIG. 15 by way of flow chart connector FC4, where the risk data visualization tool 142 displays a subject data analysis controller and a dashboard communication controller on the grid. At step 1506, the risk data visualization tool 142 of the clinical trial data analyzer 110 may detect that the user has manipulated the input device to activate the subject data analysis controller while a profile marker is highlighted on the grid. In response to the activation of the subject data analysis controller, at step 1508, the risk data visualization tool 142 automatically generates and displays on the display device a subject data table including variables, measurements for the variables, and metric values for the measurements, for a patient associated with the highlighted profile marker. The measurements may include measurements for the patient across all visits that the patient made to the clinical unit.

At step 1510, the risk data visualization tool 142 may also detect that the user has manipulated the input device to activate the dashboard communication controller while the profile marker is highlighted on the grid. In response to the activation of the dashboard communication controller, at step 1512, the risk data visualization tool 142 of the clinical trial data analyzer 110 automatically generates and displays on the display device a dashboard dialog panel configured to permit the user to create a risk alert record for the clinical unit and the variable associated with the highlighted profile marker. The risk alert record may include data fields for saving one or more of a clinical data unit identifier, a variable identifier, a risk level identifier, an action identifier, a status identifier, a metric for the variable, and a user-generated description of a risk. At step 1514, the risk alert record is stored in the risk data object 182 on the secondary memory device 118, and at step 1516 at least a portion of the risk data object 182 may be transmitted to the issue tracking system 106 for the clinical trial.

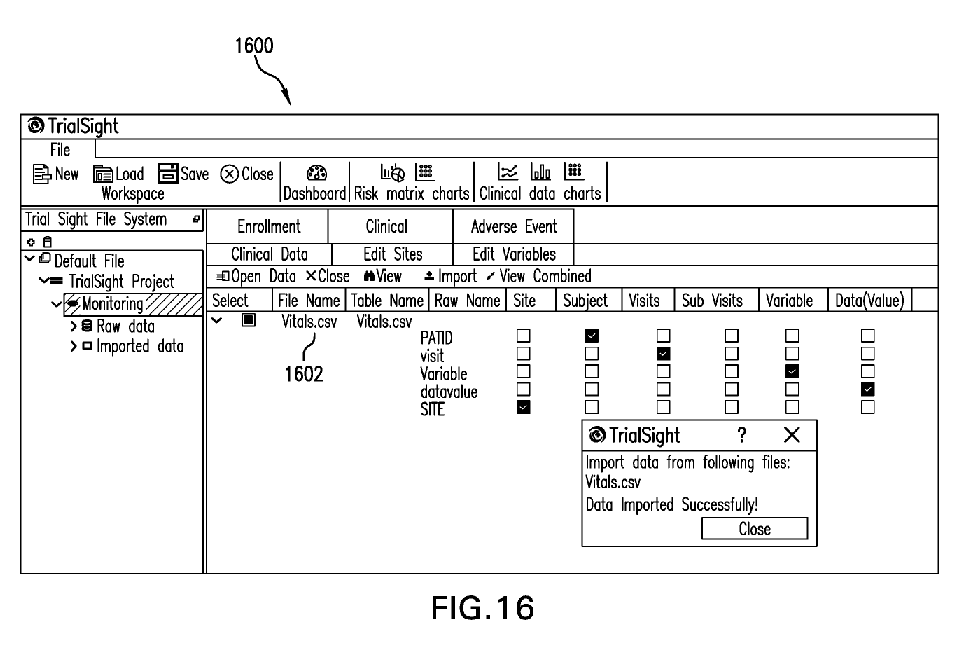

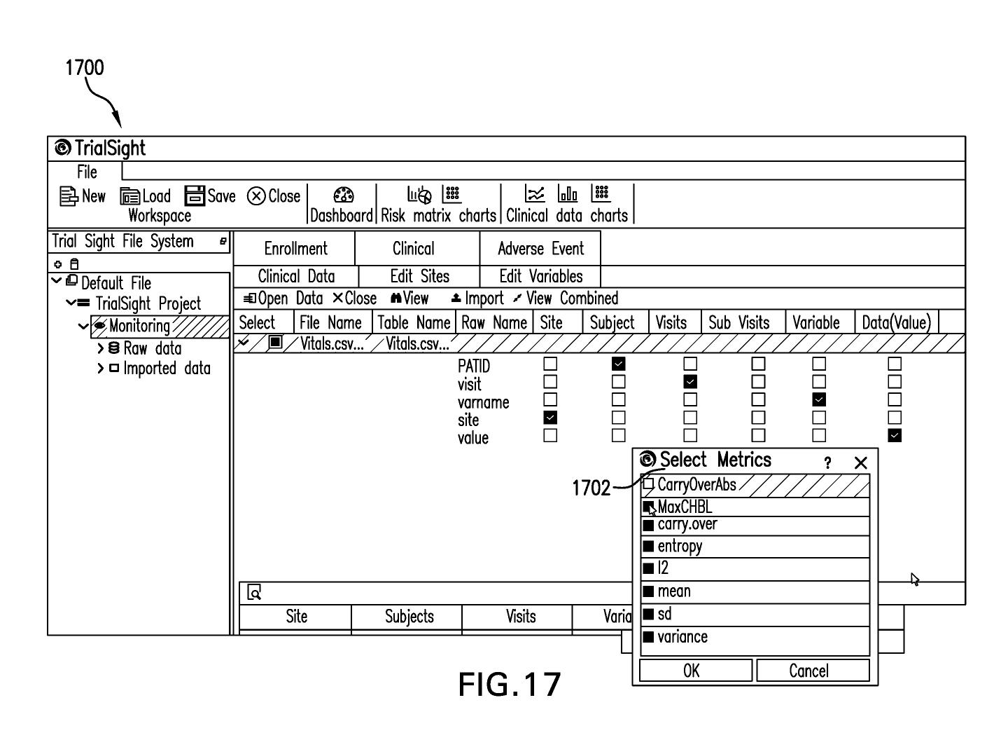

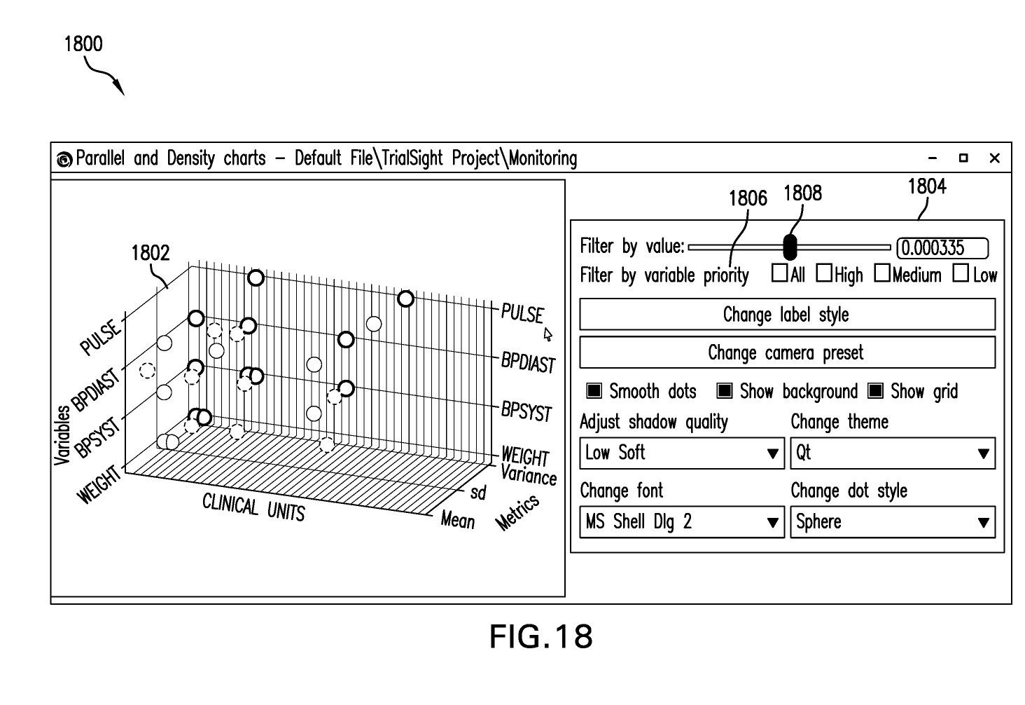

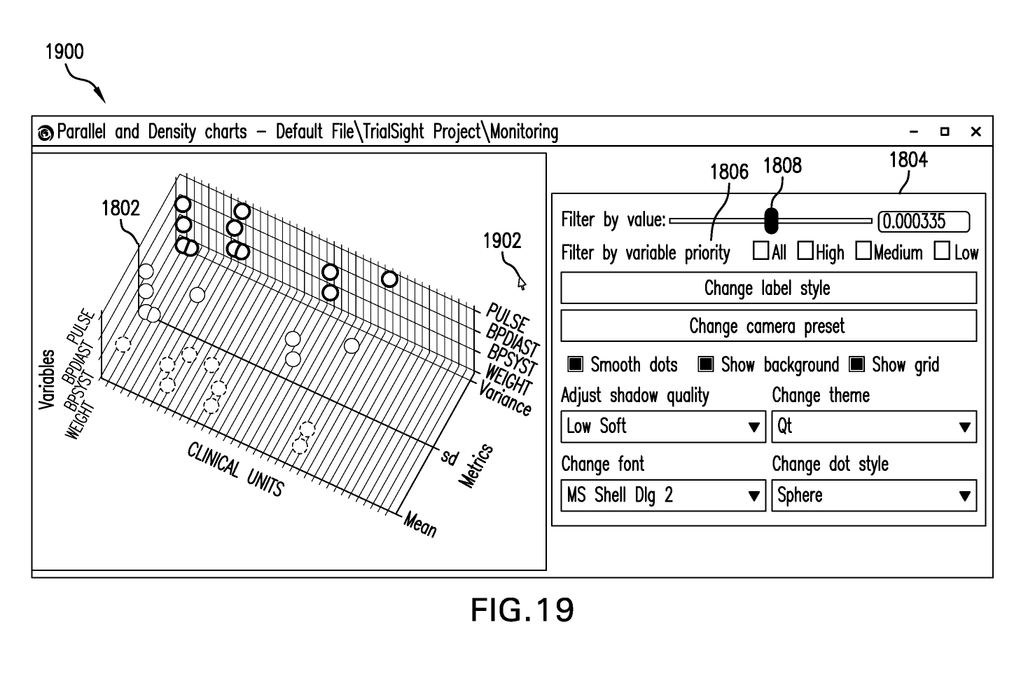

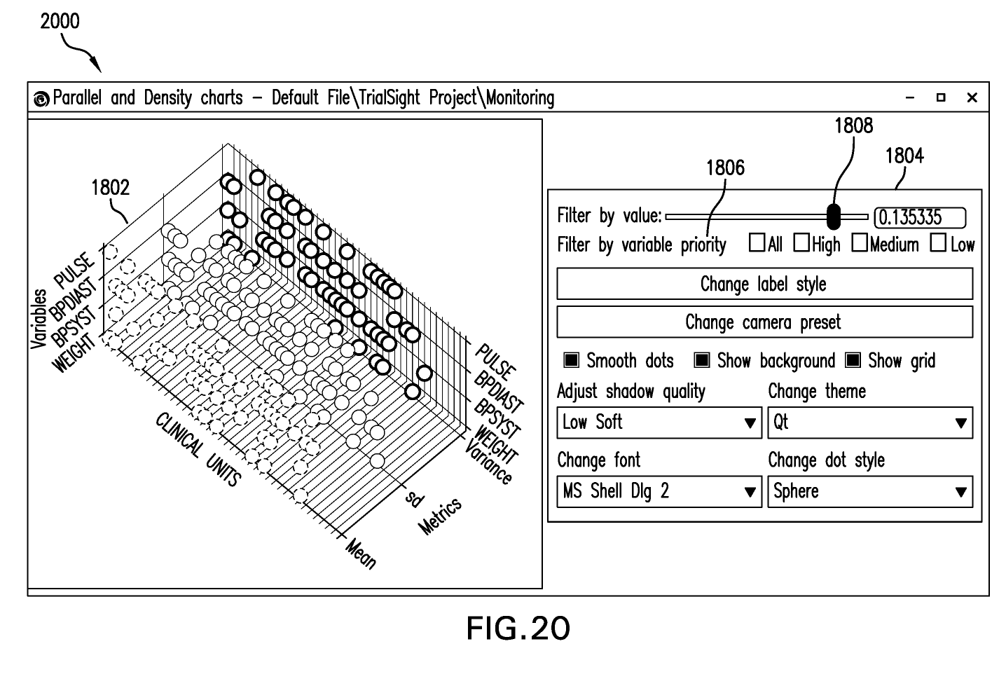

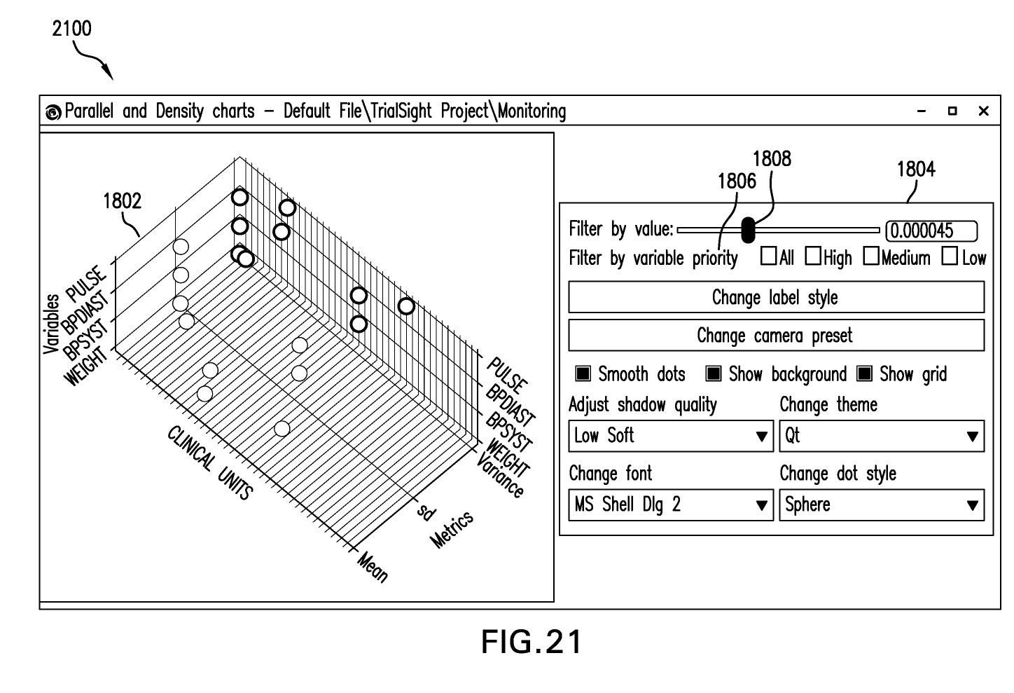

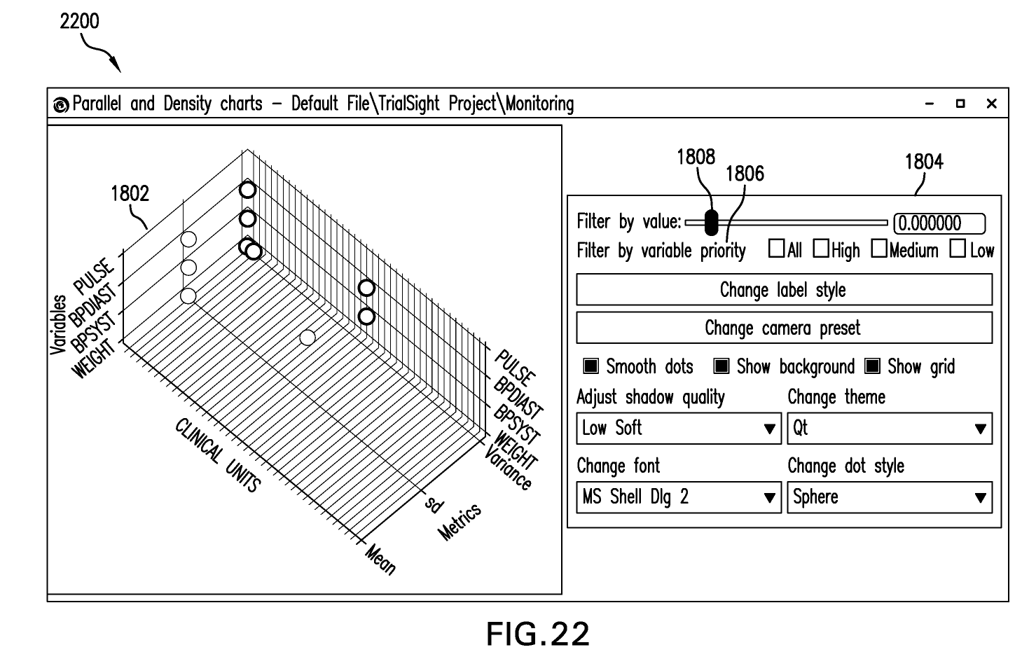

FIGS. 16-30 show additional exemplary user interface screens (i.e., screenshots 1600, 1700, 1800, 1900, and 2000, respectively) for clinical data analysis in embodiments of the present invention. The screenshot 1600 in FIG. 16 illustrates an example data import screen for selecting which clinical data to import from a “.csv” file 1602. The screenshot 1700 in FIG. 17 illustrates an example screen for selecting metric functions 1702 to be applied to clinical data. The screenshot 1800 in FIG. 18 illustrates a resulting three-dimensional risk matrix 1802 and a portion of the screen 1804 that provides a priority selector 1806 and a risk threshold controller 1808. The screenshot 1900 in FIG. 19 illustrates a subsequent screen where, in response to a user gesture on the screen (e.g., holding and dragging the mouse pointer 1902 around the screen), a three-dimensional rotation is applied to the three-dimensional matrix 1802 so that the user may view the three-dimensional matrix from a different angle. The screenshot 2000 in FIG. 20 illustrates the three-dimensional matrix 1802 from yet another angle and with a different selected risk threshold that causes more risk scores to pass the threshold and be revealed as additional signal markers. The screenshots 2100 and 2200 in FIGS. 21 and 22, respectively, illustrate the three-dimensional matrix 1802 with other risk threshold values selected (by moving the risk threshold controller 1808 on the slider) to allow fewer risk scores to pass the threshold so that more of the signal markers are concealed.

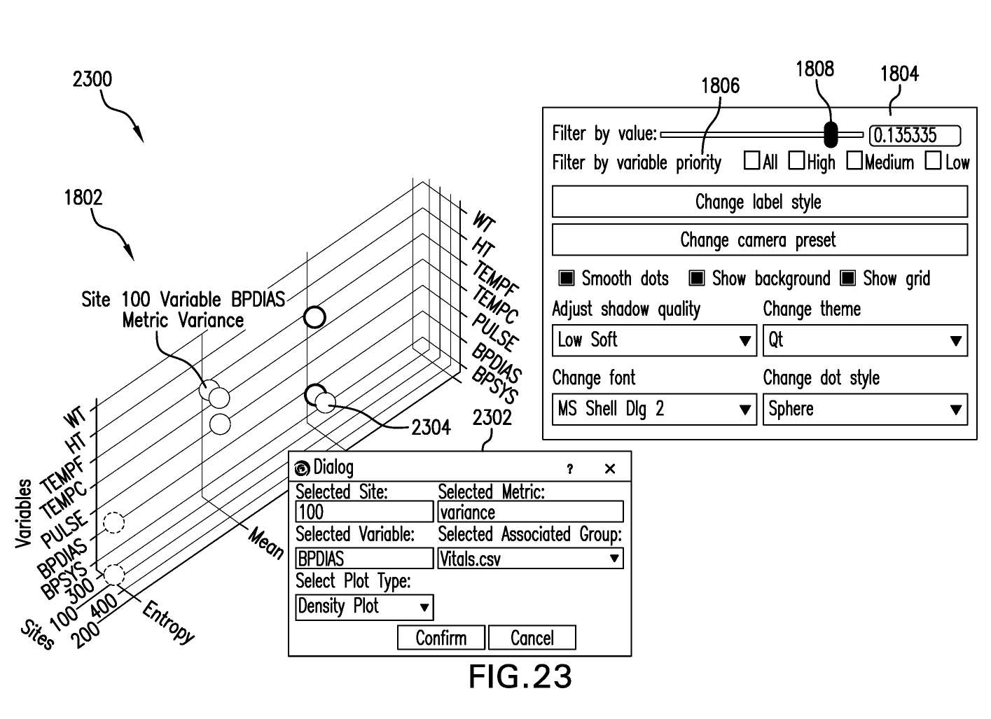

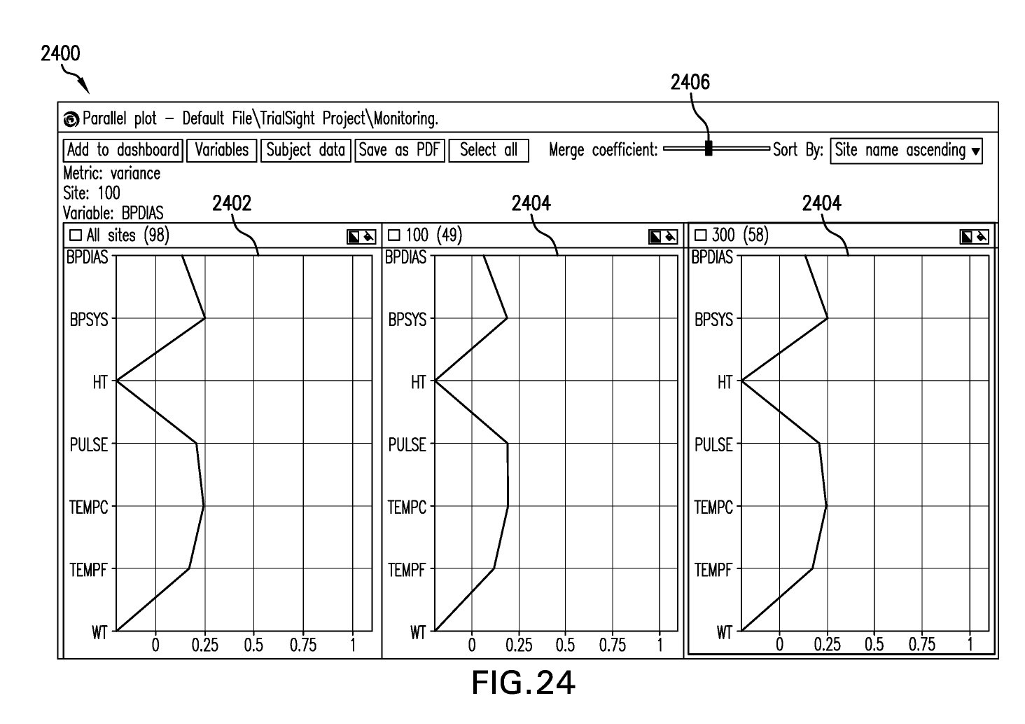

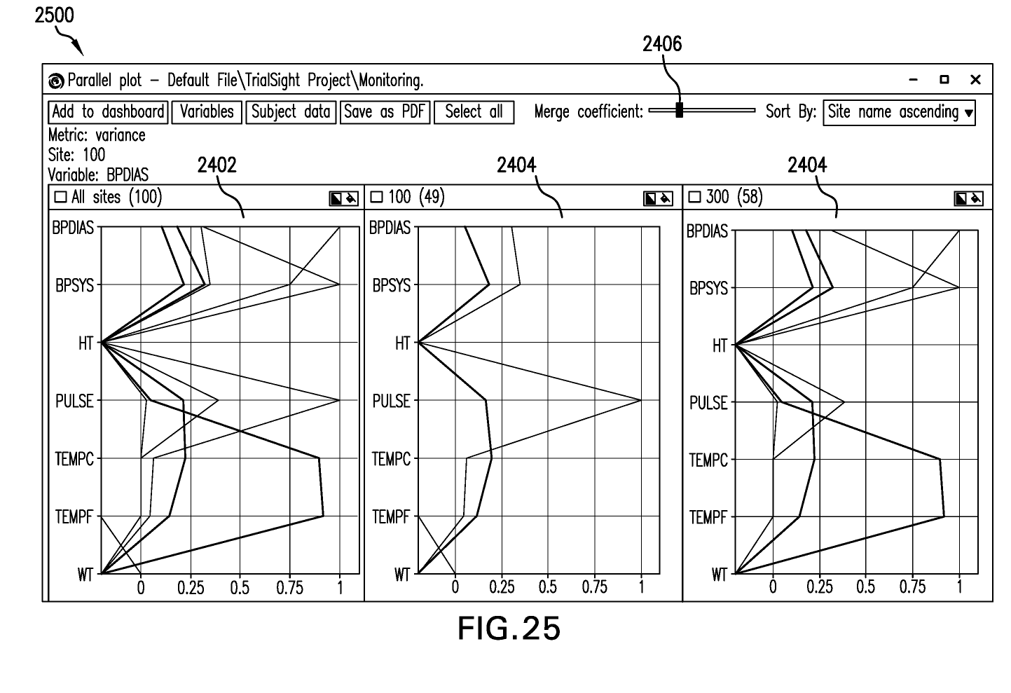

FIG. 23 shows a screenshot 2300 illustrating an investigative panel 2302 that opens when a signal marker 2304 is selected in the three-dimensional risk matrix 1802 to select a plot type for further investigation. FIG. 24 shows a screenshot 2400 illustrating a resulting grid when a parallel coordinate plot type is selected. The grid includes a combined box 2402 for all units combined and individual boxes 2404 for each unit individually. Each box includes multiple profile markers corresponding to multiple patients. The multiple profile markers in each box may be merged into fewer profile markers based on a merge coefficient selected via a clustering tool 2406 (in this case a slider). In one embodiment, the higher the coefficient, the fewer profile markers will be displayed. FIG. 25, for example, shows a screenshot 2500 illustrating the result of using a lower merge coefficient, as compared to the screenshot 2400 in FIG. 24. By using the clustering tool 2406, patients are clustered into multiple groups based on a similarity measure, so that the differences between the groups of patients will be more evident on the display screen. Selecting a profile marker of a patient group opens a box showing more detailed data for patients in that group. The grouping may be performed based on a tolerance on the distances between two profile markers. The distances may be weighted according to the variables and then combined. The weights may be selected based on an overall variability. The merging functionality in the parallel coordinate plot allows for reducing the dimensionality related to the number of patients within a unit or within all units combined.

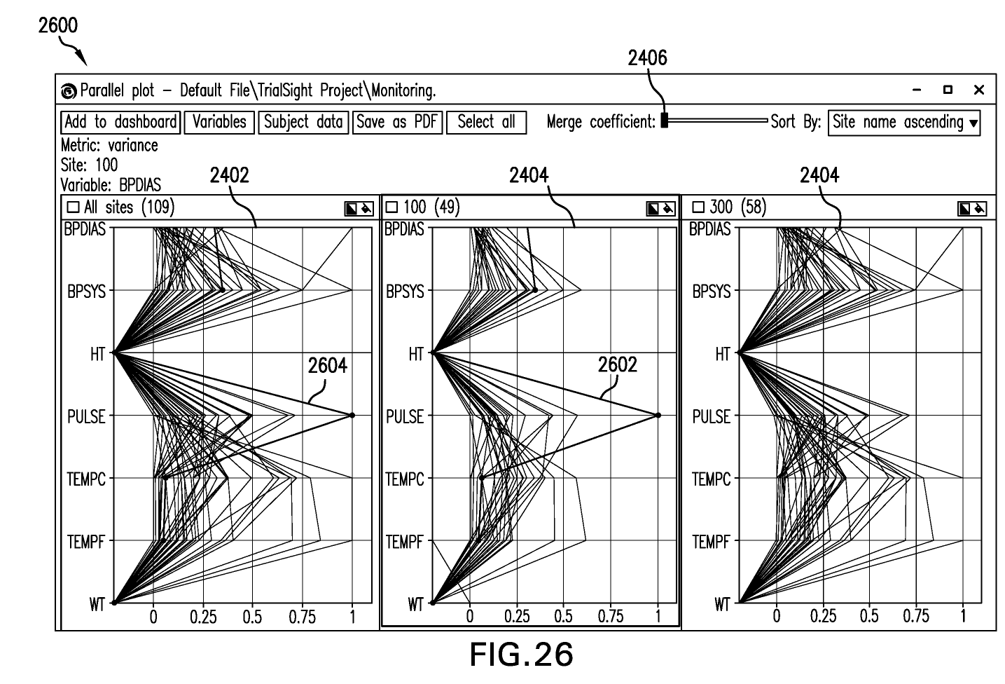

FIG. 26 shows a screenshot 2600 illustrating by way of example the result of a user selecting a particular profile marker 2602 on one of the parallel coordinate plots 2404. As shown in FIG. 26, when a particular profile marker 2602 on one of the parallel coordinate plots 2404 is selected, a profile marker 2604 on a different parallel coordinate plot 2402 that is related to the same patient is also automatically highlighted. In the parallel coordinate plots, each box (2402, 2404) provides a plot transparency control functionality. The user may change the transparency of plots in the “All Units” plot (2402) or any other plot (2404) so that the user can more easily detect which profile markers represent values that fall outside the typical range of values for these variables. The atypical profile markers are indicative of possible anomalies, while the typical profile markers indicate a possible expected behavior.

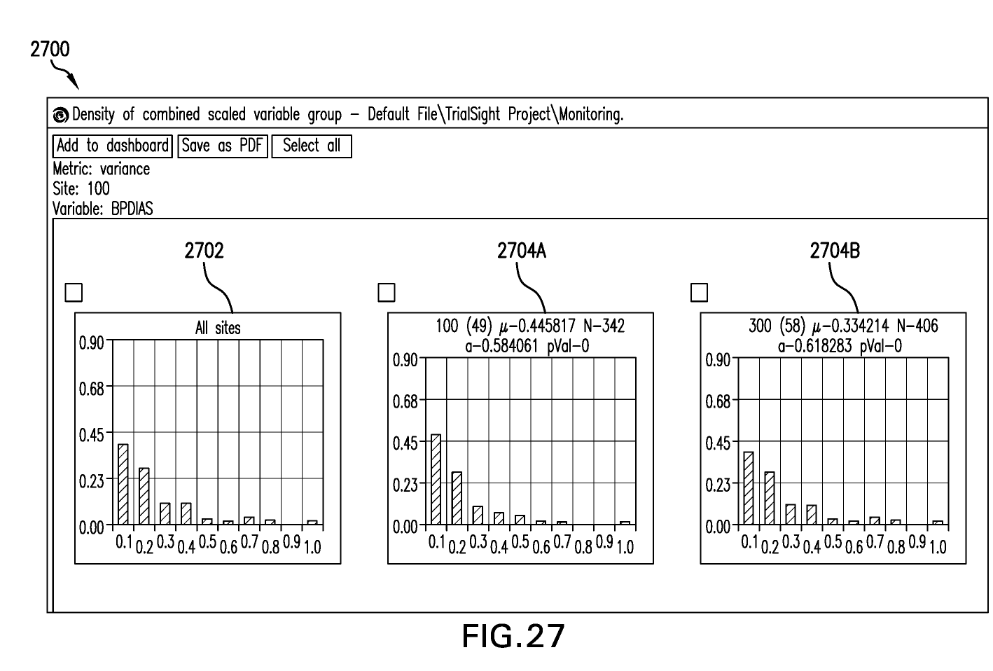

FIG. 27 shows a screenshot 2700, which illustrates by example a screen displayed after the user selects the signal marker 2304 representative of a risk score associated with function “f”, unit “u”, and variable “v” from the three-dimensional matrix 1802 in FIG. 23, and requests in the investigative panel 2302 that a combined density plot is displayed on the display device. The combined density plot includes one density plot 2702 for all units and additional density plots 2704A and 2704B for each individual clinical unit, including clinical unit “u”. Each density plot 2702, 2704A and 2704B shows the standard deviation of the metric values obtained by applying a function “f” to measurements of variable “v” (or measurements of each variable in a group of variables associated with variable “v”) in one clinical unit (density plots 2704A or 2704B) or over all units (density plot 2702). The density plot indicates whether measurements in a unit have the same variability that measurements in other units have. At the top of each individual unit density plot 2704A and 2704B, a mean and a standard deviation of the metric values displayed in that density plat may also be displayed.

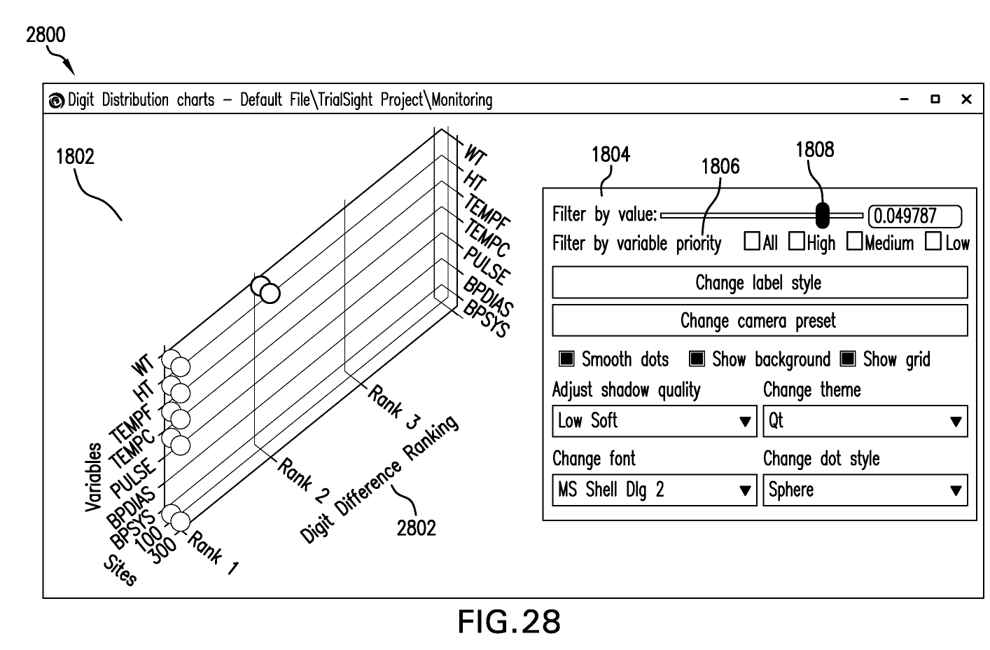

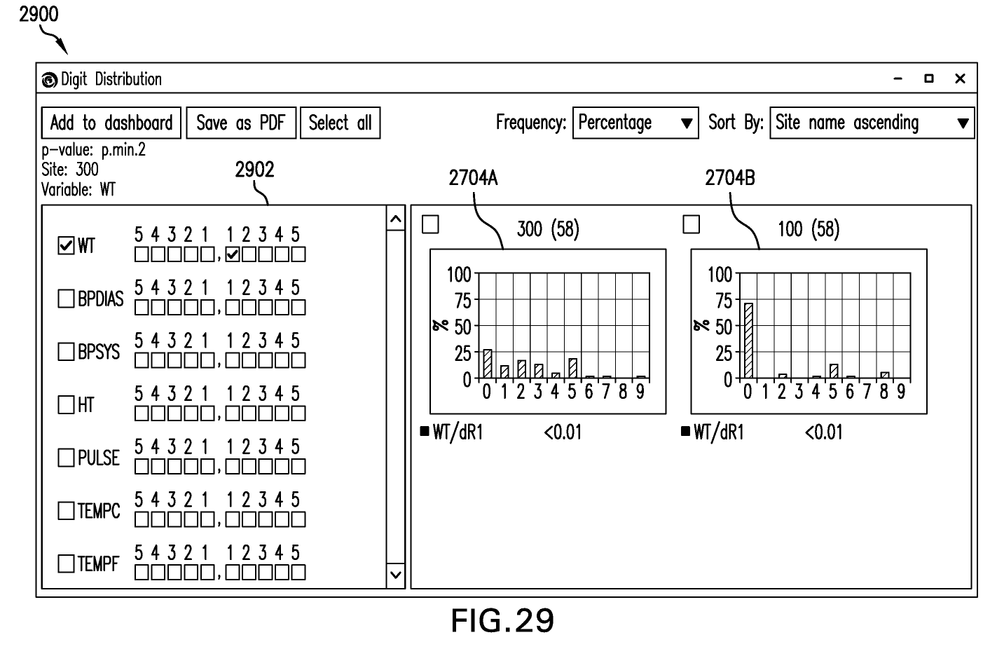

FIG. 28 shows screen shot 2800 illustrating by example how the three-dimensional risk matrix 1802 appears on the display device when the selected metric function is digit preference. As shown in FIG. 28, the distribution of every digit of every variable for a given unit is compared to the distribution across all other units. This risk matrix shows the most disparate findings, ranked in order of the most disparate to the least disparate, for every unit, variable and digit combination. The risk matrix 1802 depicted in FIG. 28 shows three ranks on the digit difference ranking axis 2802. The number of ranks shown is configurable by the human operator. In this case, the matrix reveals a symbol marker (colored ball) when the risk associated with a risk value exceeds the threshold value set by the human operator. When a symbol marker on the risk matrix 1802 shown in FIG. 28 is selected by the operator, a digit distribution matrix 2900 (shown in FIG. 29) displays the distribution of the digits in table 2902 for the variable and unit associated with the selected symbol marker. As shown in FIG. 29, the digit distribution matrix 2900 also includes a first plot 2704A for the given unit (unit 300 in this case), followed by plots for each of the other units (in this case, unit 100). While viewing this display the user can explore other digit distributions for other variables.

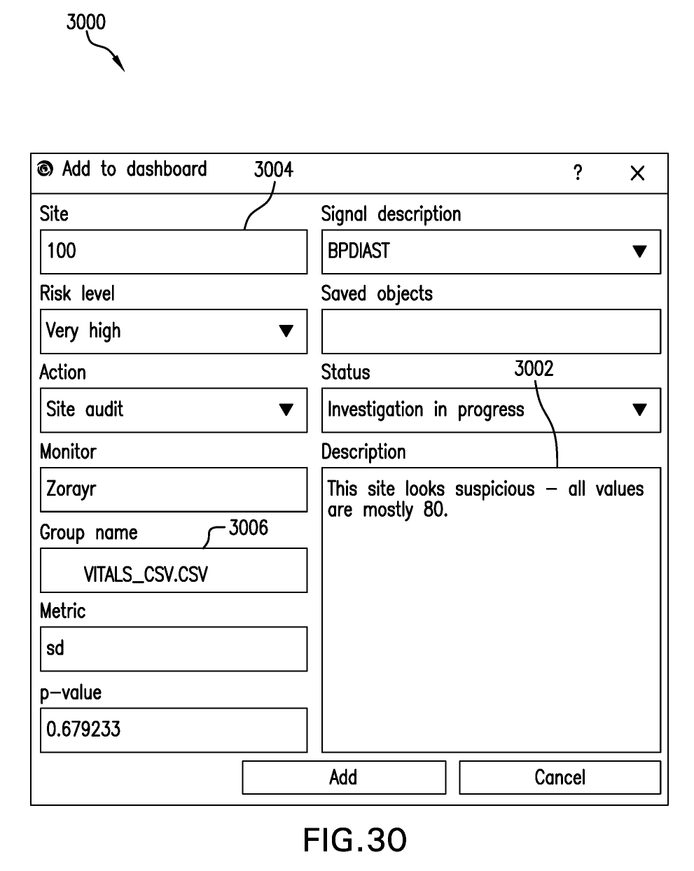

FIG. 30 shows an example of a dashboard 3000 for creating a risk alert. In this example, the dashboard 3000 is used to create a risk alert including a risk alert description 3002 indicating suspicious (e.g., potentially erroneous) measurements of a group of variables 3006 at a clinical site 3004. The dashboard may be configured to pull risk alert information from the risk data object, to push risk alert information to the risk data object, or both.

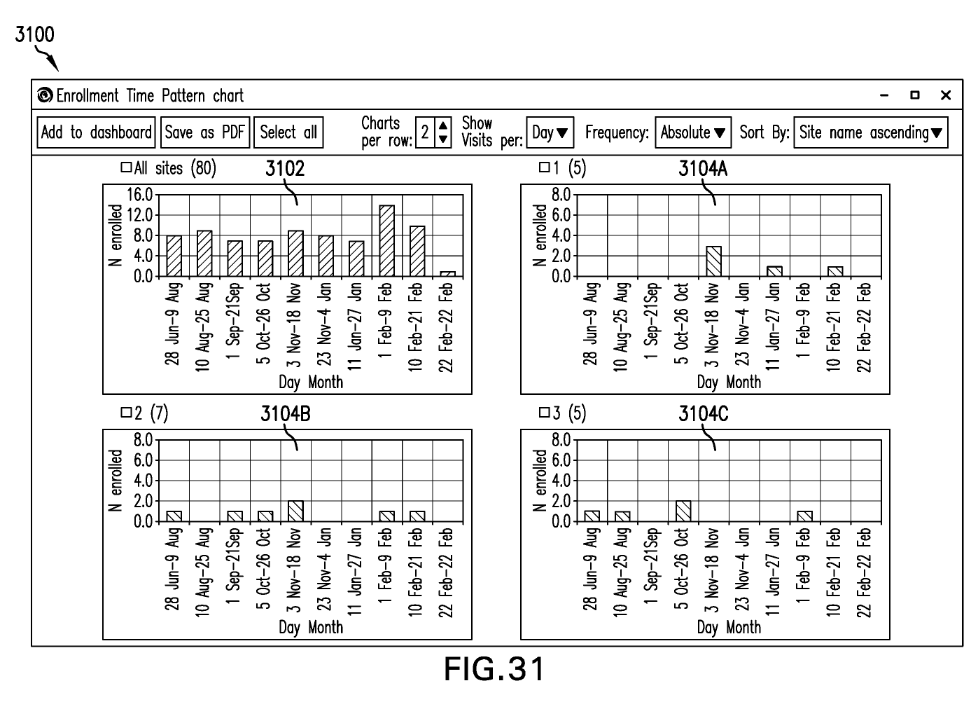

FIG. 31 shows a screen shot 3100 depicting one example of an enrollment time pattern chart that may be generated and displayed in some embodiments of the present invention. In the example shown in FIG. 31, the enrollment time pattern chart comprises four graphs, including a first graph 3102 showing the total number of participants enrolled in all the clinical units during each month of an eight-month period. The enrollment time pattern chart also includes three additional graphs 3104A, 3104B and 3104C showing the number of participants enrolled at each of three individual clinical units during each month of the same eight-month period. The enrollment time pattern chart permits the user to look for patterns suggesting errors or data issues associated with enrollment procedures used at each clinical unit, and permits the user to save those issues to the dashboard.

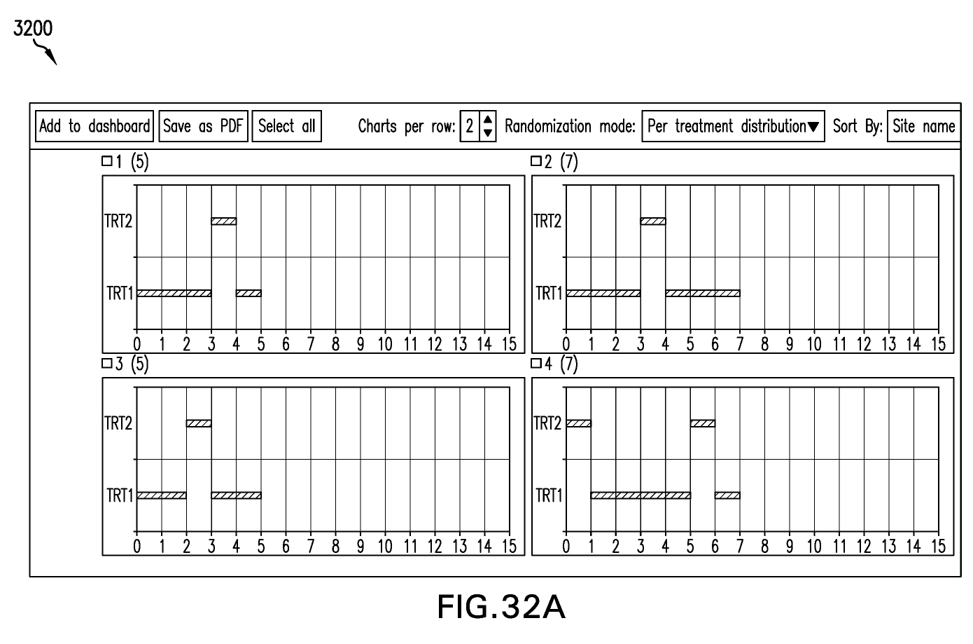

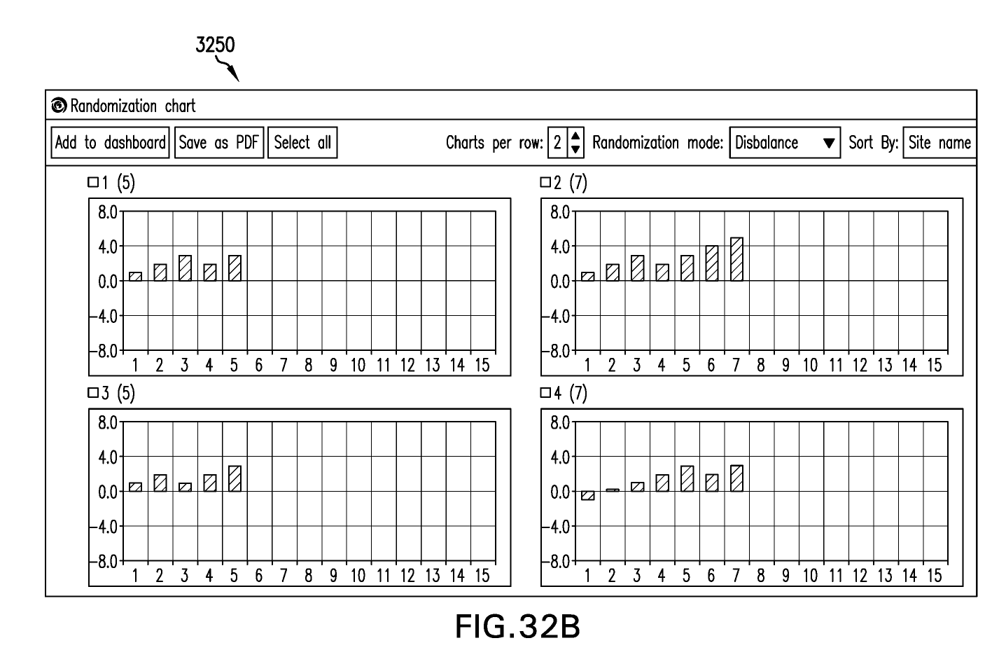

FIG. 32A shows a screen shot 3200 illustrating by example a distribution of treatment or intervention assignments when each participant is enrolled, allowing the user to look for incorrectly assigned interventions to add to the dashboard. FIG. 32B shows screen shot 3250 showing by example the distribution of assignments of one treatment or intervention relative to another treatment or intervention, allowing the user to evaluate if there is an imbalance between the two treatment or intervention assignments and when that imbalance occurs.

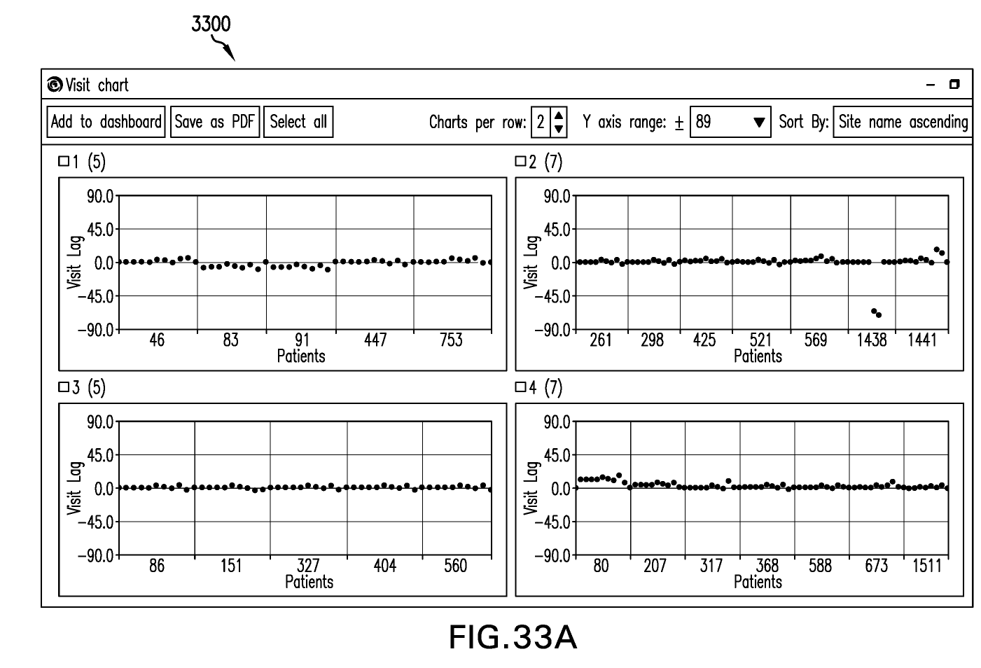

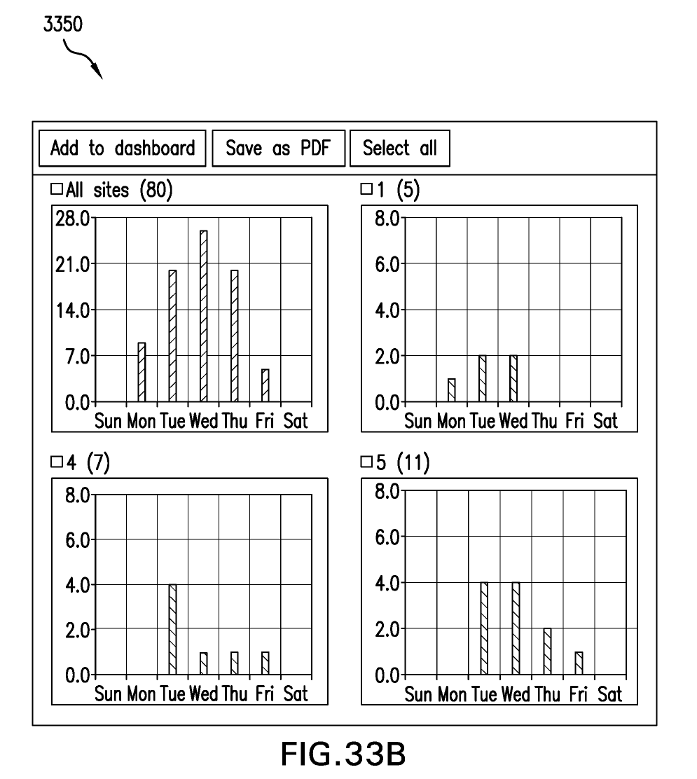

FIG. 33A shows a screen shot 3300 illustrating by example the number of days between when a visit occurred and when that visit was expected to have occurred. For example, a visit occurring exactly when expected is illustrated by a symbol on the zero axis. Exhibit 33B shows a screen shot 3350 illustrating by example the days of the week on which visits occurred allowing the users to detect unexpected events for example visits on Sundays when the unit is closed on Sundays.

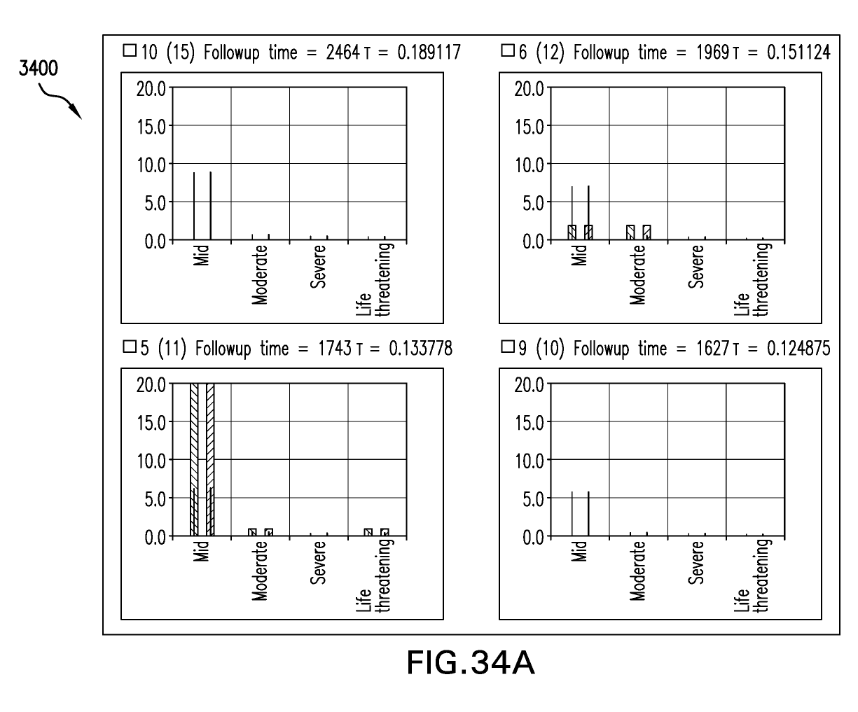

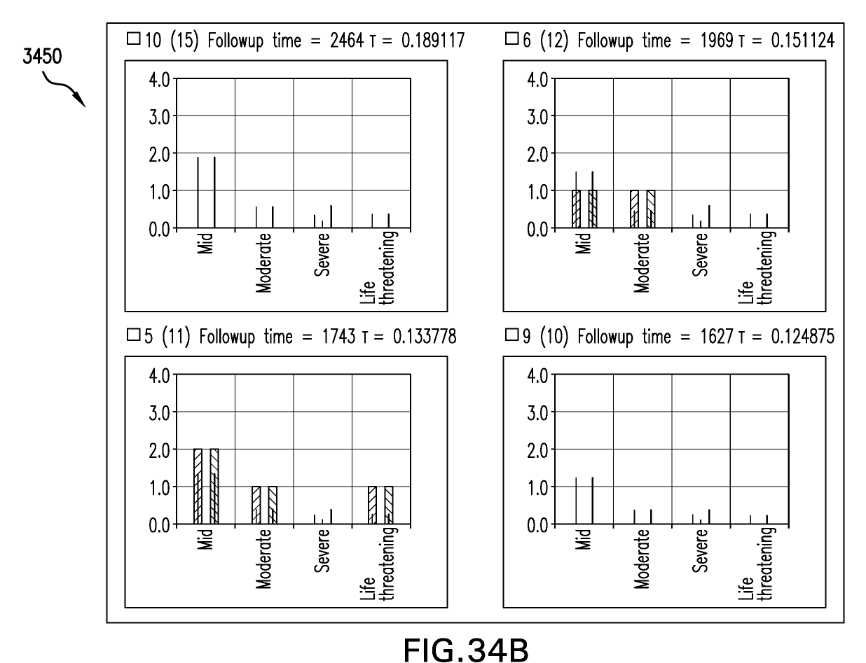

FIG. 34A shows a screen shot 3400 illustrating by example the distribution of adverse events by severity (the bars) compared to the expected distribution for that severity across all sites (the lines within the bars). The first bar within a severity category represents any adverse event not considered serious and the second bar within a severity category represents all reported adverse events regardless of seriousness. FIG. 34B shows a screen shot 3450 illustrating by example the distribution of reported events for the worst severity category ever reported for a participant so that each participant is only represented once in the visualization. Although exemplary embodiments, uses and advantages of the invention have been diclosed above with a certain degree of particularity, it will be apparent to those skilled in the art, upon consideration of this specification and practice of the invention as disclosed herein, that alterations and modifications can be made without departing from the spirt or the scope of the invention, which are intended to be limited only by the following claims and equivalents thereof.

What is claimed is:

1. A method of analyzing clinical data from a clinical trial on a computer system, the method comprising:
importing into a memory device associated with the computer system the clinical data including multiple measurements of multiple variables of multiple patients, wherein measurements of each patient are obtained during multiple visits of the patient to a single one of multiple clinical units;
storing the clinical data in a trial monitoring data object on the memory device;
using a microprocessor on the computer system to calculate multiple metric values, each metric value indicating a statistical attribute of a variable of a patient, wherein the microprocessor generates each metric value by applying a metric function to measurements of the variable of the patient collected over multiple visits by the patient to a clinical unit;
creating an analytical data object in the memory device, wherein the analytical data object stores the multiple metric values;
determining by the microprocessor, for each metric function “f” and each variable “v”, a risk score “Sf,v(u)” associated with each clinical unit “u”;
creating in the memory device a risk data object that stores the risk scores “Sf,v(u)” for all metric functions “f”, all variables “v”, and all units “u”, ; and
displaying a graphic representation of the risk scores on a display device associated with the computer system.

2. The method of claim 1, wherein each patient visits a single clinical unit within the multiple clinical units two or more times without visiting any other clinical unit in the multiple clinical units.

3. The method of claim 1, further comprising applying a mask to the trial monitoring data object by the microprocessor to hide or ignore, for purposes of a current analytical session, any clinical data associated with known issues, known problems, or known risks that are not germane to a current area of investigation.

4. The method of claim 1, wherein the multiple metric functions comprise a statistical function.

5. The method of claim 4, wherein the statistical function comprises at least one of a standard deviation, an entropy, a mean value, an average value, a rate of identical measurements, a sum of distances from neighboring visits, and an occurrence of similar within tolerance measurements.

6. The method of claim 1, wherein, for a metric function “f1”, a variable “v1”, and a clinical unit “u1”, a risk score “S_{f1,v1}(u1)” indicates a relative strength of differences between the clinical unit “u1” as compared to all other clinical units based on metric values obtained by applying the metric function “f1” to measurements of the variable “v1”.

7. The method of claim 6, wherein the higher the risk score “S_{f1,v1}(u1)” is, the more different the clinical unit “u1” is from other clinical units as indicated by metric values “MV_{f1,v1}(pat)” of all patients “pat” of all units.

8. The method of claim 6, wherein the lower the risk score “S_{f1,v1}(u1)” is, the more different the clinical unit “u1” is from other clinical units as indicated by metric values “MV_{f1,v1}(pat)” of all patients “pat” of all units.

9. The method of claim 6, wherein the risk score “S_{f1,v1}(u1)” indicates the likelihood of an error or data issue in measurements of the variable “v1” at the clinical unit “u1” given measurements of same or related variables at other units.

10. The method of claim 6, wherein the risk score “S_{f1,v1}(u1)” is obtained by applying a risk scoring function to the metric values obtained by applying the metric function “f1” to the measurements of the variable “v1” at the clinical unit “u1”.

11. The method of claim 10, wherein the risk scoring function assigns the risk score “S_{f1,v1}(u1)” based on a comparison of a collection of metric values associated with the clinical unit “u1” with a collection of metric values associated with all other units combined.

12. The method of claim 10, wherein the risk scoring function measures a magnitude of a probabilistic difference between a collection of metric values associated with the clinical unit “u1” with a collection of metric values associated with all other units combined.

13. The method of claim 10, wherein the risk scoring function comprises hypothesis testing.

14. The method of claim 13, wherein the hypothesis testing is based on a null hypothesis that a probabilistic distribution of a collection of metric values associated with the clinical unit “u1” is not different, beyond a given threshold, than a probabilistic distribution of a collection of metric values associated with all other units combined.

15. The method of claim 13, wherein the hypothesis testing is Bayesian.

16. The method of claim 13, wherein the hypothesis testing uses a parametric framework that depends on a type of the variable “v1” or a type of the metric function “f1”.

17. The method of claim 16, wherein the parametric model is binomial when the metric function “f1” is a rate of occurrence.

18. The method of claim 16, wherein the parametric model is Chi-square when the metric function “f1” is standard deviation.

19. The method of claim 6, wherein the risk score “S_{f1,v1}(u1)” comprises a P-value from a statistical test, wherein the P-value is the probability of observing metric values at least as extreme as metric values obtained by applying the metric function “f1” to measurements of the variable “v1” at the clinical unit “u1”, assuming that there are no differences between the clinical unit “u1” and other clinical units.

20. The method of claim 1, wherein the step of displaying the graphic representation of the risk scores comprises causing the microprocessor to use the contents of the risk data object to display a three-dimensional risk matrix on the display device, wherein the three-dimensional risk matrix includes a selection of clinical units on a first axis, a selection of variables on a second axis, and a selection of metric functions on a third axis.

21. The method of claim 20, wherein the selection of variables includes a group of related variables.

22. The method of claim 20, further comprising displaying on the display device a variable selector, a priority selector, and a risk threshold controller, wherein the variable selector is operable by a user to configure the selection of variables, the priority selector is operable by the user to select a priority level for variables, and the risk threshold selector is operable by the user to select a current risk threshold for risk scores associated with the selection of metric functions.

23. The method of claim 22, further comprising the step of automatically displaying on the display device a signal marker in the three-dimensional risk matrix to represent each combination of variable/unit/function of “v1”, “u1”, and “f1” for which “v1” is in the selection of variables, and “v1” has a variable priority greater than or equal to a variable priority selected by the priority selector, and “f1” is in the selection of functions, and risk score “S_{f1,v1}(u)” is greater than or equal to the current risk threshold.

24. The method of claim 23, further comprising the step of automatically concealing from view on the display device every signal marker in the three-dimensional risk matrix that represents a combination of variable/unit/function of “v2”, “u2”, and “f2” for which “v2” is not in the selection of variables, or “v2” has a variable priority less than the variable priority selected by the priority selector, or “f2” is not in the selection of functions, or risk score “S_{f2,v2}(u2)” is less than the current risk threshold.

25. The method of claim 22, further comprising the step of automatically displaying on the display device a signal marker in the three-dimensional risk matrix to represent each combination of variable/unit/function of “v1”, “u1”, and “f1” for which “v1” is in the selection of variables, and “v1” has a variable priority greater than or equal to a variable priority selected by the priority selector, and “f1” is in the selection of functions, and risk score “S_{f1,v1}(u)” is less than or equal to the current risk threshold.

26. The method of claim 25, further comprising the step of automatically concealing from view on the display device every signal marker in the three-dimensional risk matrix that represents a combination of variable/unit/function of “v2”, “u2”, and “f2” for which “v2” is not in the selection of variables, or “v2” has a variable priority less than the variable priority selected by the priority selector, or “f2” is not in the selection of functions, or risk score “Sf2,v2(u2)” is greater than the current risk threshold.

27. The method of claim 24, further comprising automatically displaying additional signal markers or automatically concealing additional signal markers when the user operates at least one of the variable selector, the priority selector, or the risk threshold selector on the display device to change a variable selection, to change a priority or to change a risk threshold.

28. The method of claim 27, further comprising:
detecting by the computer system that the user has manipulated an input controller associated with the computer system to select a first signal marker displayed on the three-dimensional risk matrix on the display device;
using the risk data object stored on the memory device to identify a combination of variable/unit/function of “v3”, “u3”, and “f3” represented by the selected signal marker; and
generating and displaying an investigation panel on the display device, the investigation panel being configured to prompt the user to select or confirm at least one of “v3”, “u3”, “f3”, a variable group associated with “v3”, or a plot type to use for root cause analysis of data represented by the selected signal marker.

29. The method of claim 28, further comprising generating and displaying on the display device a grid comprising multiple plots rendered in accordance with the selected plot type.

30. The method of claim 29, wherein the plot type comprises a parallel coordinate plot, wherein the grid includes a combined plot for all clinical units combined, as well as individual plots for each clinical unit including “u3”, wherein the combined plot illustrates metric values obtained by applying “f3” to the variables in the variable group in all clinical units combined, and each individual plot illustrates metric values obtained by applying “f3” to the variables in the variable group only for one clinical unit.

31. The method of claim 30, further comprising:
detecting that the user has manipulated the input controller to select a first profile marker in the combined plot; and
visually highlighting the first profile marker in the combined plot and automatically visually highlighting a second profile marker on a second plot on the grid on the display device, wherein the first profile marker and the second profile marker are associated with data of the same patient collected at the same clinical unit.

32. The method of claim 31, further comprising providing a subject data analysis controller and a dashboard communication controller on the grid on the display device.

33. The method of claim 32, further comprising detecting that the user has manipulated the input device to activate the subject data analysis controller while a profile marker is highlighted on the grid on the display device.

34. The method of claim 33, further comprising automatically generating and displaying on the display device a subject data table including variables, measurements for the variables, and metric values for the measurements, for a patient associated with the highlighted profile marker.

35. The method of claim 33, further comprising detecting that the user has manipulated the input device to activate the dashboard communication controller while the profile marker is highlighted on the grid.

36. The method of claim 35, further comprising automatically generating and displaying on the display device a dashboard dialog panel configured to permit the user to create a risk alert record for the clinical unit and the variable associated with the highlighted profile marker.

37. The method of claim 36, wherein the risk alert record comprises data fields for saving one or more of a clinical data unit identifier, a variable identifier, a metric for the variable, and a user-generated description of a risk.

38. The method of claim 36, further comprising storing the risk alert record in the risk data object in the memory device.

39. The method of claim 38, further comprising transmitting at least a portion of the risk data object to a remote issue tracking system for the clinical trial.

40. A computer system for analyzing clinical data from a clinical trial, the computer system comprising:
a primary memory device;
a secondary memory device;
a display device;
a microprocessor; and
an application program, stored on the primary memory device, comprising programming instructions that, when executed by the microprocessor, will cause the microprocessor to:
import into the secondary memory device the clinical data including multiple measurements of multiple variables of multiple patients, wherein measurements of each patient are obtained during multiple visits of the patient to a single one of the multiple clinical units;
store the clinical data in a trial monitoring data object in the secondary memory device;
apply multiple metric functions to measurements of the variable of the patient collected over multiple visits to a clinical unit to generate multiple metric values, each metric value indicating a statistical attribute of a variable of a patient;
create an analytical data object in the secondary memory device, wherein the analytical data object stores the metric values;
determine, for each metric function “f” and each variable “v”, a risk score “S_{f,v}(u)” associated with each clinical unit “u”;
create in the secondary memory device a risk data object that stores the risk scores “S_{f,v}(u)” for all metric functions “f”, all variables “v”, and all units “u”; and
display a graphic representation of the risk scores on the display device.

41. The computer system of claim 40, wherein each patient visits a single clinical unit within the multiple clinical units two or more times without visiting any other clinical unit in the multiple clinical units.

42. The computer system of claim 40, wherein the multiple metric functions include at least one of a standard deviation, an entropy, a mean value, an average value, a rate of identical measurements, a sum of distances from neighboring visits, and an occurrence of similar measurements.

43. The computer system of claim 40, wherein, for a metric function “f1”, a variable “v1”, and a clinical unit “u1”, a risk score “S_{f1,v1}(u1)” indicates a relative strength of differences between the clinical unit “u1” as compared to all other units based on metric values obtained by applying the metric function “f1” to measurements of the variable “v1”.

44. The computer system of claim 43, wherein the risk score “S_{f1,v1}(u1)” indicates the likelihood of an error in the measurements of the variable “v1” at the clinical unit “u1” given measurements of same or related variables at other units.

45. The computer system of claim 43, wherein the application program further comprises programming instructions that, when executed by the microprocessor, will cause the microprocessor to assign a risk score “S_{f1,v1}(u1)” by applying a risk scoring function to the metric values obtained by applying the metric function “f1” to the measurements of the variable “v1”.

46. The computer system of claim 45, wherein the application program further comprises programming instructions that, when executed by the microprocessor, will cause the microprocessor to assign the risk score “S_{f1,v1}(u1)” based on a comparison of a collection of metric values associated with the clinical unit “u1” with a collection of metric values associated with all other units combined.

47. The computer system of claim 45, wherein the application program further comprises programming instructions that, when executed by the microprocessor, will cause the microprocessor to measure a magnitude of a probabilistic difference between a collection of metric values associated with the clinical unit “u1” with a collection of metric values associated with all other units combined.

48. The computer system of claim 40, wherein the application program further comprises programming instructions that, when executed by the microprocessor, will cause the microprocessor to use contents of the risk data object to display a three-dimensional risk matrix on the display device, the three-dimensional risk matrix including a selection of clinical units on a first axis, a selection of variables on a second axis, and a selection of metric functions on a third axis.

49. The computer system of claim 48, wherein the application program further comprises programming instructions that, when executed by the microprocessor, will cause the microprocessor to display on the display device a variable selector, a priority selector, and a risk threshold controller, wherein the variable selector is operable by a user to configure the selection of variables, the priority selector is operable by the user to select a priority level for variables, and the risk threshold selector is operable by the user to select a current risk threshold for risk scores associated with the selection of metric functions.

50. The computer system of claim 49, wherein the application program further comprises programming instructions that, when executed by the microprocessor, will cause the microprocessor to automatically display on the display device a signal marker in the three-dimensional risk matrix to represent each combination of variable/unit/function of “v1”, “u1”, and “f1” for which “v1” is in the selection of variables, and “v1” has a variable priority greater than or equal to a variable priority selected by the priority selector, and “f1” is in the selection of functions, and risk score “S_{f1,v1}(u)” is greater than or equal to the current risk threshold.

51. The computer system of claim 50, wherein the application program further comprises programming instructions that, when executed by the microprocessor, will cause the microprocessor to automatically conceal from view on the display device every signal marker in the three-dimensional risk matrix that represents a combination of variable/unit/function of “v2”, “u2”, and “f2” for which “v2” is not in the selection of variables, or “v2” has a variable priority less than the variable priority selected by the priority selector, or “f2” is not in the selection of functions, or risk score “S_{f2,v2}(u2)” is less than the current risk threshold.

52. The computer system of claim 49, wherein the application program further comprises programming instructions that, when executed by the microprocessor, will cause the microprocessor to automatically display on the display device a signal marker in the three-dimensional risk matrix to represent each combination of variable/unit/function of “v1”, “u1”, and “f1” for which “v1” is in the selection of variables, and “v1” has a variable priority greater than or equal to a variable priority selected by the priority selector, and “f1” is in the selection of functions, and risk score “S_{f1,v1}(u)” is less than or equal to the current risk threshold.

53. The computer system of claim 52, wherein the application program further comprises programming instructions that, when executed by the microprocessor, will cause the microprocessor to automatically conceal from view on the display device every signal marker in the three-dimensional risk matrix that represents a combination of variable/unit/function of “v2”, “u2”, and “f2” for which “v2” is not in the selection of variables, or “v2” has a variable priority less than the variable priority selected by the priority selector, or “f2” is not in the selection of functions, or risk score “S_{f2,v2}(u2)” is greater than the current risk threshold.

54. The computer system of claim 51, wherein the application program further comprises programming instructions that, when executed by the microprocessor, will cause the microprocessor to automatically reveal or conceal additional signal markers when the user operates at least one of the variable selector, the priority selector, or the risk threshold selector on the display device to change a variable selection, to change a priority selection or to change a risk threshold.

55. The computer system of claim 54, wherein the application program further comprises programming instructions that, when executed by the microprocessor, will cause the microprocessor to:
detect that the user has manipulated an input controller associated with the computer system to select a first signal marker displayed on the three-dimensional risk matrix on the display device;
use the risk data object stored on the secondary memory device to identify a combination of variable/unit/function of “v3”, “u3”, and “f3” represented by the selected signal marker; and
generate and display an investigation panel on the display device, the investigation panel being configured to prompt the user to select or confirm at least one of “v3”, “u3”, “f3”, a variable group associated with “v3”, or a plot type to use for root cause analysis of data represented by the selected signal marker.

56. The computer system of claim 55, wherein the application program further comprises programming instructions that, when executed by the microprocessor, will cause the microprocessor to generate and display on the display device a grid comprising multiple plots rendered in accordance with the selected plot type.

57. The computer system of claim 56, wherein the plot type comprises parallel coordinate plot, wherein the grid includes a combined plot for all clinical units combined, as well as individual plots for each clinical unit including “u3”, wherein the combined plot illustrates metric values obtained by applying “f3” to the variables in the variable group in all clinical units combined, and each individual plot illustrates metric values obtained by applying “f3” to the variables in the variable group only for one clinical unit.

58. The computer system of claim 57, wherein the application program further comprises programming instructions that, when executed by the microprocessor, will cause the microprocessor to:
detect that the user has manipulated the input controller to select a first profile marker in the combined plot; and
visually highlight the first profile marker in the combined plot and automatically visually highlight a second profile marker on a second plot on the grid on the display device, wherein the first profile marker and the second profile marker are associated with the data of the same patient collected at the same clinical unit.

59. The computer system of claim 58, wherein the application program further comprises programming instructions that, when executed by the microprocessor, will cause the microprocessor to display on the display device a subject data analysis controller and a dashboard communication controller on the grid.

60. The computer system of claim 59, wherein the application program further comprises programming instructions that, when executed by the microprocessor, will cause the microprocessor to detect that the user has manipulated the input device to activate the subject data analysis controller while a profile marker is highlighted on the grid on the display device, and automatically generate and display on the display device a subject data table including variables, measurements for the variables, and metric values for the measurements, for a patient associated with the highlighted profile marker.

61. The computer system of claim 59, wherein the application program further comprises programming instructions that, when executed by the microprocessor, will cause the microprocessor to detect that the user has manipulated the input device to activate the dashboard communication controller while the profile marker is highlighted on the grid, and automatically generate and display on the display device a dashboard dialog panel configured to permit the user to create a risk alert record for the clinical unit and the variable associated with the highlighted profile marker.

62. The computer system of claim 61, wherein the risk alert record comprises data fields for saving one or more of a clinical data unit identifier, a variable identifier, a metric for the variable, and a user-generated description of a risk, wherein the microprocessor is further configured to store the risk alert record in the risk data object in the secondary memory device and transmit at least a portion of the risk data object to a remote issue tracking system for the clinical trial.

63. A computer system for analyzing clinical data from a clinical trial, the clinical data including multiple measurements of multiple variables of multiple patients obtained during multiple visits of the multiple patients to multiple clinical units, the computer system comprising:
a display device;
a microprocessor;
a primary memory device for storing an application program comprising instructions executable by the microprocessor;
a secondary memory device for storing a trial monitoring data object, an analytical data object, and a risk data object, wherein the trial monitoring data object stores the clinical data, the analytical data object stores metric values for each variable of each patient, and the risk data object stores risk scores “S_{f,v}(u)” for each metric function “f”, variable “v”, and unit “u”;
wherein the application program includes instructions that, when executed by the microprocessor, will cause the microprocessor to:
automatically generate each metric value stored in the analytical data object by applying one of multiple metric functions to measurements of a variable of a patient collected over multiple visits to a clinical unit to indicate a statistical attribute of the measurements of the variable of the patient, and
automatically generate the risk scores “S_{f,v}(u)” for each metric function “f”, variable “v”, and unit “u” based on a comparison of a collection of metric values associated with unit “u” with a collection of metric values associated with all other units combined, and
display on the display device a graphic representation of the risk scores.

64. The computer system of claim 63, wherein each patient visits a single clinical unit within the multiple clinical units two or more times without visiting any other clinical unit in the multiple clinical units.

65. The computer system of claim 63, wherein the multiple metric functions comprise a statistical function, wherein the statistical function comprises at least one of a standard deviation, an entropy, a mean value, an average value, a rate of identical measurements, a sum of distances from neighboring visits, and an occurrence of similar within tolerance measurements.

66. The computer system of claim 63, wherein, for a metric function “f1”, a variable “v1”, and a clinical unit “u1”, a risk score “S_{f1,v1}(u1)” indicates a relative strength of differences between the clinical unit “u1” as compared to all other units based on metric values obtained by applying the metric function “f1” to measurements of the variable “v1”.

67. The computer system of claim 66, wherein the risk score “S_{f1,v1}(u1)” indicates the likelihood of an error in the measurements of the variable “v1” at the clinical unit “u1” given measurements of same or related variables at other units.

68. The computer system of claim 63, wherein the application program includes instructions that, when executed by the microprocessor, will cause the microprocessor to use contents from the risk data object to display a three-dimensional risk matrix on the display device, wherein the three-dimensional risk matrix includes a selection of clinical units on a first axis, a selection of variables on a second axis, and a selection of metric functions on a third axis.

69. The computer system of claim 68, wherein the application program includes instructions that, when executed by the microprocessor, will cause the microprocessor to display a variable selector, a priority selector, and a risk threshold controller on the display device, wherein the variable selector is operable by a user to configure the selection of variables, wherein the priority selector is operable by the user to select a priority level for variables, and wherein the risk threshold selector is operable by the user to select a current risk threshold for risk scores associated with the selection of metric functions.

70. The computer system of claim 69, wherein the application program includes instructions that, when executed by the microprocessor, will cause the microprocessor to automatically display on the display device a signal marker in the three-dimensional risk matrix to represent each combination of variable/unit/function of “v1”, “u1”, and “f1” for which “v1” is in the selection of variables, and “v1” has a variable priority greater than or equal to a variable priority selected by the priority selector, and “f1” is in the selection of functions, and risk score “S_{f1,v1}(u)” is greater than or equal to the current risk threshold.

71. The computer system of claim 70, wherein the application program includes instructions that, when executed by the microprocessor, will cause the microprocessor to automatically conceal from view on the display device every signal marker in the three-dimensional risk matrix that represents a combination of variable/unit/function of “v2”, “u2”, and “f2” for which “v2” is not in the selection of variables, or “v2” has a variable priority less than the variable priority selected by the priority selector, or “f2” is not in the selection of functions, or risk score “Sf2,v2(u2)” is less than the current risk threshold.

72. The computer system of claim 69, wherein the application program includes instructions that, when executed by the microprocessor, will cause the microprocessor to automatically display on the display device a signal marker in the three-dimensional risk matrix to represent each combination of variable/unit/function of “v1”, “u1”, and “f1” for which “v1” is in the selection of variables, and “v1” has a variable priority greater than or equal to a variable priority selected by the priority selector, and “f1” is in the selection of functions, and risk score “S_{f1,v1}(u)” is less than or equal to the current risk threshold.

73. The computer system of claim 72, wherein the application program includes instructions that, when executed by the microprocessor, will cause the microprocessor to automatically conceal from view on the display device every signal marker in the three-dimensional risk matrix that represents a combination of variable/unit/function of “v2”, “u2”, and “f2” for which “v2” is not in the selection of variables, or “v2” has a variable priority less than the variable priority selected by the priority selector, or “f2” is not in the selection of functions, or risk score “S_{f2,v2}(u2)” is greater than the current risk threshold.

74. The computer system of claim 71, wherein the application program includes instructions that, when executed by the microprocessor, will cause the microprocessor to automatically reveal or conceal additional signal markers when the user operates at least one of the variable selector, the priority selector, or the risk threshold selector on the display device.

75. The computer system of claim 74, wherein the application program includes instructions that, when executed by the microprocessor, will cause the microprocessor to:
detect that the user has manipulated an input controller associated with the computer system to select a first signal marker displayed on the three-dimensional risk matrix on the display device;
use the risk data object to identify a combination of variable/unit/function of “v3”, “u3”, and “f3” represented by the selected signal marker; and
generate and display an investigation panel on the display device, the investigation panel being configured to prompt the user to select or confirm at least one of “v3”, “u3”, “f3”, a variable group associated with “v3”, or a plot type to use for root cause analysis of data represented by the selected signal marker.

76. The computer system of claim 75, wherein the application program includes instructions that, when executed by the microprocessor, will cause the microprocessor to generate and display on the display device a grid comprising multiple plots rendered in accordance with the selected plot type.

77. The computer system of claim 76, wherein the plot type comprises parallel coordinate plot, wherein the grid includes a combined plot for all clinical units combined, as well as individual plots for each clinical unit including “u3”, wherein the combined plot illustrates metric values obtained by applying “f3” to the variables in the variable group in all clinical units combined, and each individual plot illustrates metric values obtained by applying “f3” to the variables in the variable group only for one clinical unit.

78. The computer system of claim 77, wherein the application program includes instructions that, when executed by the microprocessor, will cause the microprocessor to:
detect that the user has manipulated the input controller to select a first profile marker in the combined plot; and
visually highlight the first profile marker in the combined plot and automatically visually highlight a second profile marker on a second plot on the grid on the display device, wherein the first profile marker and the second profile marker are associated with the data of the same patient collected at the same clinical unit.

79. The computer system of claim 78, wherein the application program includes instructions that, when executed by the microprocessor, will cause the microprocessor to display a subject data analysis controller and a dashboard communication controller on the grid on the display device.

80. The computer system of claim 79, wherein the application program includes instructions that, when executed by the microprocessor, will cause the microprocessor to detect that the user has manipulated the input device to activate the subject data analysis controller while a profile marker is highlighted on the grid on the display device, and automatically generate and display on the display device a subject data table including variables, measurements for the variables, and metric values for the measurements, for a patient associated with the highlighted profile marker.

81. The computer system of claim 79, wherein the application program includes instructions that, when executed by the microprocessor, will cause the microprocessor to detect that the user has manipulated the input device to activate the dashboard communication controller while the profile marker is highlighted on the grid on the display device, and automatically generate and display on the display device a dashboard dialog panel configured to permit the user to create a risk alert record for the clinical unit and the variable associated with the highlighted profile marker.

82. The computer system of claim 81, wherein the risk alert record comprises data fields for saving one or more of a clinical data unit identifier, a variable identifier, a metric for the variable, and a user-generated description of a risk, wherein the application program includes instructions that, when executed by the microprocessor, will cause the microprocessor to store the risk alert record in the risk data object and transmit at least a portion of the risk data object to a remote issue tracking system for the clinical trial.
